(19) United States
(12) Patent Application Publication
(10) Pub. No.: US 2024/0067714 A1
(43) Pub. Date: Feb. 29, 2024

(54) FAB SIALYLATION OF ANTIBODIES

(71) Applicant: LIMMATECH BIOLOGICS AG, Schlieren (CH)

(72) Inventors: Manuela MALLY, Schlieren (CH); Amirreza FARIDMOAYER, Schlieren (CH); Rainer FOLLADOR, Schlieren (CH); Jonathan Albert BACK, Schlieren (CH)

(73) Assignee: LIMMATECH BIOLOGICS AG, Schlieren (CH)

(21) Appl. No.: 18/025,792

(22) PCT Filed: Sep. 13, 2021
(86) PCT No.: PCT/EP2021/075076

§ 371 Date: Mar. 10, 2023

Related U.S. Application Data
(60) Provisional application No. 63/078,218, filed on Sep. 14, 2020.

(51) Int. Cl. C07K 16/24

(52) U.S. Cl. CPC C07K 16/241; A61K 2039/545; C07K 2317/14; C07K 2317/41; C07K 2317/52; C07K 2317/55; C07K 2317/94

(57) ABSTRACT

The present disclosure provides monoclonal antibodies (e.g., anti-TNFα antibodies) having Fab sialylation, and, in some aspects, Fab sialylation in combination with an afucosylated Fc. Such antibodies having improved immunogenicity profiles and related advantages. Such glycan modifications will improve current treatments and allow a better quality of life for patients. Accordingly, such monoclonal antibodies are useful for treating and preventing various diseases.

This application claims priority to U.S. Provisional Application No. 63/078,218, filed on Sep. 14, 2020, the entirety of which is incorporated herein by reference.

### SEQUENCE LISTING

This application incorporates by reference in its entirety the Computer Readable Format (CRF) of a Sequence Listing in ASCII text format. The Sequence Listing text file is entitled “14197-013-228_Seqlisting ST25,” was created on Sep. 10, 2021 and is 11,632 bytes in size.

### FIELD

The present invention relates generally to antibodies having sialylation of the Fab region of the antibody, and, in some aspects, Fab sialylation in combination with an afucosylated Fc.

### BACKGROUND

A significant proportion of patients treated with biopharmaceuticals develop unwanted immune reactions against the drug that can be detrimental to treatment. The development of anti-drug antibodies (ADA) is one of the biggest challenges in the sustained effectiveness of current biological therapies. Monoclonal antibodies constitute a large portion of marketed biological therapies. In addition to target binding, an antibody also interacts with receptors on immune cells thereby contributing to immune-regulation. The regulation of these effector functions is in part directed by a set of conserved N-linked glycans on the Fc-part of the antibody.

Additionally, in the context of inflammatory bowel disease (IBD), due to the unwanted immune reactions in the form of ADA against anti-TNFα, up to 38% of patients lose treatment response (Vermeire et al., Therap Adv Gastroenterol., 2018; 11:1756283X17750355, Published 2018 Jan. 21, doi:10.1177/1756283X17750355). Reduction of the ADA response will clearly improve patient care and quality of life.

The compositions and methods provided herein address the unmet medical need of patients suffering from various diseases treated with anti-TNFα, such as IBD, and provide related advantages.

### SUMMARY

The present disclosure provides monoclonal antibodies having sialylation of the Fab region of the antibody. Accordingly, in some embodiments, such antibodies have a higher amount of sialic acid in the Fab region of the monoclonal antibody as compared to a control antibody, such as an antibody found in human serum or a monoclonal antibody produced by a Chinese hamster ovary (CHO) cell line. The present disclosure also provides monoclonal antibodies having sialylation of the Fc region of the antibodies. Accordingly, in some embodiments, such antibodies have a higher amount of sialic acid in a Fab region and/or a higher amount of sialic acid in an Fc region of the monoclonal antibody as compared to a control antibody, such as an antibody found in human serum or a monoclonal antibody produced by a CHO cell line.

The present disclosure also provides monoclonal antibodies having an afucosylated glycan in the Fc region of the antibody. Accordingly, in some embodiments, such antibodies have a higher amount of sialic acid in a Fab region and/or a higher amount of afucosylated glycan in an Fc region of the monoclonal antibody as compared to a control antibody, such as an antibody found in human serum or a monoclonal antibody produced by a CHO cell line.

The present disclosure also provides monoclonal antibodies having a G0 glycan in an Fc region of the antibody. Accordingly, in some embodiments, such antibodies have a higher amount of sialic acid in a Fab region and/or higher amount of G0 glycan in an Fc region of the monoclonal antibody as compared to a control antibody, such as an antibody found in human serum or a monoclonal antibody produced by a CHO cell line.

The monoclonal antibodies provided herein can also be engineered antibodies (e.g., variants of a known antibody) to provide for sites for glycosylation. Accordingly, in some embodiments, such antibodies have sialylated glycans at one or more point mutations in the variable domain of the heavy chain and/or light chain of the monoclonal antibody and/or sialylated glycans at one or more inserted or mutated amino acids leading to an N-glycosylation site in the framework region of the variable domain of the heavy chain of the monoclonal antibody, while such antibodies retain their ability to bind their antigen.

The present disclosure also provides herein a host cell for production of such monoclonal antibodies and methods of making such monoclonal antibodies. Accordingly, in some embodiments, provided herein is a Leishmania host cell, such as Leishmania tarentolae. In some embodiments, provided herein is a method of making a monoclonal antibody provided herein by culturing the Leishmania host cell and isolating the monoclonal antibody.

The present disclosure also provides pharmaceutical compositions and methods of using the monoclonal antibodies provided herein to treat or prevent a disease. Accordingly, in some embodiments, provided herein is a pharmaceutical composition having a monoclonal antibody described herein and pharmaceutically acceptable carrier. Also provided herein, in some embodiments, is a single dosage form of a monoclonal antibody provided herein. In some embodiments, provided herein is a method of treating or preventing a disease in a patient that includes administering to the patient a monoclonal antibody described herein or a pharmaceutical composition described herein. Diseases that can be treated or prevented using the methods described herein include an inflammatory bowel disease, such as Crohn's disease, pediatric Crohn's disease, ulcerative colitis, microscopic colitis, diverticulosis-associated colitis, collagenous colitis, lymphocytic colitis, or Behcet's disease, or other inflammatory diseases, such as rheumatoid arthritis, psoriatic arthritis, ankylosing spondylitis, chronic psoriasis, hidradenitis suppurativa, adult uveitis, pediatric uveitis, plaque psoriasis, or juvenile idiopathic arthritis.

### BRIEF DESCRIPTION OF THE DRAWINGS

FIG. 1 shows N-glycans of the Fab glycosylation site of adalimumab K84N (top) with high abundance of alpha 2,6 biantennary sialylation and N-glycans of conserved N-glycosite 297 in the Fc part (bottom) after expression in St19788.

FIG. 2 shows N-glycans of the Fab glycosylation site of adalimumab K84N-D86N (top) with high abundance of alpha 2,6 biantennary sialylation and N-glycans of conserved N-glycosite 297 in the Fc part (bottom) after expression in St19790.

FIG. 3 shows N-glycan relative abundance of antibodies expressed in different CGP cell line background.

FIGS. 4A-4B show Fab sialylated glycans are accessible and recognized by SNA lectin, in contrast to Fc sialylated glycans. FIG. 4A shows the ELISA assay principle. FIG. 4B shows the average±SD of duplicates measurements of absorbance values for each conditions.

FIGS. 5A-5B show that Fab-sialylated adalimumab displays a reduced immunogenicity, whereas Fc-sialylated adalimumab does not consistently show reduced immunogenicity.

FIG. 6 shows that Fab-sialylated adalimumab variants enhance mucosal healing M2 macrophages induction.

FIGS. 7A-7B show that Fab-sialylated adalimumab variants have higher ADCC activity than HUMIRA in a standard assay.

FIG. 8 shows that Fab-sialylated adalimumab variants have ADCC activity against primary target cells expressing physiological levels of TNF, in contrast to HUMIRA.

FIG. 9 shows N-glycans of the Fab glycosylation site of adalimumab K84N-D86N (top) with high abundance of alpha 2,6 biantennary sialylation and N-glycans of conserved N-glycosite 297 in the Fc part (middle) after expression in St19866 in comparison with the N-glycans of the conserved N-glycosite 297 in the Fc part of HUMIRA (bottom).

FIG. 10 shows binding affinities of adalimumab A-8486S (dark grey) and HUMIRA (light grey) to a panel of Fc receptors. Affinity values for FcγRIIA, FcγRIIB, FcRn were estimated using a steady state model. Affinity values for FcγRT, FcγRIIIA and FcγRIIIB were estimated using a heterogenous ligand model yielding in two K_{D} values with the first one being the more meaningful one. Fold changes were calculated K_{D}(HUMIRA)/K_{D}(A-8486S).

FIG. 11 shows N-glycans of the Fab glycosylation site of adalimumab K84N-D86N (top) with high abundance of alpha 2,6 biantennary sialylation and N-glycans of conserved N-glycosite 297 in the Fc part (bottom) after expression in StCGP02824.

FIG. 12 shows N-glycans of the Fab glycosylation site of adalimumab K84N-D86N (top) with high abundance of alpha 2,6 biantennary sialylation and N-glycans of conserved N-glycosite 297 in the Fc part (bottom) after expression in StCGP02826.

FIG. 13 shows N-glycan relative abundance of antibodies expressed in cell lines StCGP02824 and StCGP02826.

FIG. 14 shows stability of N-glycan relative abundance of antibodies expressed in cell lines StCGP02824 and StCGP02826, by comparing the profiles after passage 4 (P4) and passage 6 (P6).

FIG. 15 shows N-glycan relative abundance of antibodies expressed in cell lines StCGP02824 and StCGP02826 in shake flask cultures (SF) compared to fed-batch fermentations (Fermenter).

FIG. 16 shows N-glycan relative abundance upon increased expression of adalimumab. Antibodies were expressed in cell lines StCGP02824 (1xA-8486) or the respective yield increased version StCGP02944 (2xA-8486) and StCGP02826 (1xA-8486) or the respective yield increased version StCGP02946 (2xA-8486).

FIG. 17 shows that only A-84S and A-8486S variants, but not the variant with sialylated N-glycan in the Fc fragment only (Fc_A-S) could bind specifically to the CHO-hCD22 cells as readout by flow cytometry. CHO cells stably expressing human CD22 (CHO-hCD22) and parental CHO-K1 cells were stained with Antibody-TNF immune complexes at 10 μg/ml, detected with APC anti-human IgG Fc secondary antibody. The specific CHO-CD22 staining intensity was calculated as the MFI CHO-hCD22—MFI CHO-K1. The X-axis of the graph shows the specific CHO-hCD22 staining intensity for indicated antibodies. A-84S indicates an adalimumab variant with a sialylated N-glycan at position 84 of the variable heavy chain; A-86S indicates an adalimumab variant with a sialylated N-glycan at position 86 of the variable light chain; A-8486S indicates an adalimumab variant with sialylated N-glycan at position 86 of the variable light chain and position 84 of the variable heavy chain; Fc_A-S indicates an adalimumab in which the Fc N-glycan (position N-297) is sialylated, this variant does not contain Fab N-glycans.

FIG. 18 shows the Monkey anti-drug antibodies (ADA) data. Cynomolgus female monkey were administered with either Humira or A-8486S (6 animals per group) at 3 mg/kg on day 1, 8 and 15 by i.v route. ADA levels against Humira and A-8486S was measured using an electrochemiluminescence based bridging assay at day 36 and day 64 (study termination). The left graph show the ADA levels at day 36 and right graph shows the ADA levels at study termination (day 64). RLU represent the relative luminescence unit (ECL signal). Each point represents the ADA level in one animal (black circles Humira and open squares A-8486S). The mean±SEM of each group are represented by the horizontal and vertical lines.

### DETAILED DESCRIPTION

Described herein is a monoclonal antibody (e.g., an anti-TNFα monoclonal antibody) having improved functionalities as compared to a control antibody (e.g., adalimumab). As exemplified herein, the adalimumab protein sequence is engineered by introduction of N-glycosylation sites in the Fab region of the antibody, resulting an engineered glycosylation profile that minimizes or even prevents ADA responses and thereby increases sustained treatment response. By customizing the N-glycan site, the engineered adalimumab variants described herein have: 1) an the afucosylated Fc N-glycan at the conserved N297 of the heavy chain of IgG1 that is expected to provide an increased primary treatment response through induction of M2 macrophages and ADCC against primary T cells; and/or 2) an biantennary sialylated glycan (“G2S2”) at the Fab glycosites that is expected to engage sialic acid binding receptors related to dampening of the immune response and leading to reduction of ADA.

Without being bound by theory, terminally sialylated glycans in the Fab domain of an anti-TNFα antibody (e.g., adalimumab), as described herein, are expected to reduce ADA development in IBD and related therapies. Moreover, the afucosylated N-glycan at the conserved N297 of the heavy chain of IgG1 is expected to provide an increased primary treatment response through induction of M2 macrophages and ADCC against primary T cells. The exposed sialic acid on N-glycans at glycosites introduced to the framework regions (FR) of the Fab domain, on either HC or LC or the combination thereof, is also expected to engage sialic acid binding receptors related to dampening of the undesired immune response. Also, several glycosites with exposed sialic acid in the Fab domain of adalimumab will further potentiate the anti-inflammatory and anti-immunogenic effect.

Additionally, there is currently limited possibilities for efficient and customized glycosylation on recombinant monoclonal antibodies. The CGP expression and glycoengineering described herein enables the generation of completely afucosylated Fc glycans for enhanced effector functions (e.g., ADCC) and highly alpha 2,6 sialylated glycan on exposed N-glycosites positioned on the Fab domain of an IgG1. The combination of Fc-afucosylation and high Fab sialylation enables the combination of unprecedented modes of action (MoA), as exemplified herein, that allows to improve patient care in IBD treatments.

The term “fragment antigen-binding” or “Fab” when used in reference to a region of an antibody (e.g., a monoclonal antibody) refers to the region of the antibody that binds to a target antigen and comprises of one constant and one variable domain of each of the heavy and light chains.

The term “fragment crystallizable” or “Fc” when used in reference to a region of an antibody (e.g., a monoclonal cantibody) refers to the region of the antibody that interacts with cell surface receptors (Fc receptors) and proteins of the complement system, which in an IgG format is comprised of two heavy chain constant domains (CH2 and CD3), and, in an IgM and IgE format is comprises of three heavy chain constant domains (CH2, CH3 and CH4).

The term “about,” when used in conjunction with a number, refers to any number within ±1, ±5 or ±10% of the referenced number.

As used herein, the term “subject” refers to an animal (e.g., birds, reptiles, and mammals). In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, a subject is a non-human animal. In some embodiments, a subject is a farm animal or pet (e.g., a dog, cat, horse, goat, sheep, pig, donkey, or chicken). In a specific embodiment, a subject is a human. The terms “subject” and “patient” may be used herein interchangeably.

The abbreviations “α[number]”, “α[number], [number]”, “β[number]”, or “β[number], [number]” refer to glycosidic bonds or glycosidic linkages which are covalent bonds that join a carbohydrate residue to another group. An α-glycosidic bond is formed when both carbons have the same stereochemistry, whereas a β-glycosidic bond occurs when the two carbons have different stereochemistry.

As used herein, a capitalized drug name represents the antibody in the brand-name drug sold under the trademark and the antibody in any biosimilar thereof, for example HUMIRA, AMJEVITA, CYLTEZO, REMICADE, SIMPONI, CIMZIA, and ENBREL. For example, HUMIRA represents the antibody adalimumab in the drug sold under the trademark HUMIRA and the antibody in any biosimilar thereof.

As used herein, the term “pharmaceutically acceptable” means approved by a regulatory agency of the Federal or a state government or listed in the U.S. Pharmacopeia or other generally recognized pharmacopeiae for use in animals, and more particularly in humans.

The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

In some embodiments, provided herein is a monoclonal antibody comprising a higher amount of sialic acid in the Fab region of the monoclonal antibody as compared to an antibody found in human serum or a control monoclonal antibody produced by a CHO cell line.

In some embodiments, provided herein is a monoclonal antibody comprising a higher amount of sialic acid in a Fab region and/or a higher amount of sialic acid in an Fc region of the monoclonal antibody as compared to an antibody found in human serum or a control monoclonal antibody produced by a CHO cell line.

In some embodiments, provided herein is a monoclonal antibody comprising a higher amount of sialic acid in a Fab region and/or a higher amount of afucosylated glycan in an Fc region of the monoclonal antibody as compared to an antibody found in human serum or a control monoclonal antibody produced by a CHO cell line.

In some embodiments, provided herein is a monoclonal antibody comprising a higher amount of sialic acid in a Fab region and/or higher amount of G0 glycan in an Fc region of the monoclonal antibody as compared to an antibody found in human serum or a control monoclonal antibody produced by a CHO cell line.

In some embodiments, provided herein is a monoclonal antibody comprising sialylated glycans at one or more point mutations in the variable domain of the heavy chain and/or light chain of the monoclonal antibody.

In some embodiments, provided herein is a monoclonal antibody comprising sialylated glycans at one or more inserted or mutated amino acids leading to an N-glycosylation site in the framework region of the variable domain of the heavy chain of the monoclonal antibody, wherein the monoclonal antibody retains its ability to bind its antigen.

In some embodiments, provided herein is a monoclonal antibody comprising sialylated glycans at one or more inserted or mutated amino acids leading to an N-glycosylation site in the framework region of the variable domain of the light chain of the monoclonal antibody, wherein the monoclonal antibody retains its ability to bind its antigen.

In some embodiments, provided herein is a monoclonal antibody comprising sialylated glycans at one or more inserted or mutated amino acids leading to an N-glycosylation site in the framework region of the variable domain of the heavy chain and sialylated glycans at one or more inserted or mutated amino acids leading to an N-glycosylation site in the framework region on the variable domain of the light chain, wherein the monoclonal antibody retains its ability to bind its antigen.

In some embodiments, provided herein is a monoclonal antibody comprising afucosylated glycan structures at the Fc region of the monoclonal antibody.

In some embodiments, provided herein is a monoclonal antibody comprising afucosylated glycan structures at the conserved Fc glycosite N297.

In some embodiments, a monoclonal antibody provided herein is an anti-TNFα antibody. Any anti-TNFα antibody known in the art can be used as the monoclonal antibody described herein. In a specific embodiment, the anti-TNFα antibody is the anti-TNFα antibody of Homo sapiens. In some embodiments, the anti-TNFα antibody is a full length antibody, an Fab, an F(ab′)2, an Scfv, or a sdAb. In a specific embodiment, the anti-TNFα antibody is a full length antibody, an Fab, an F(ab′)2, an Scfv, or a sdAb of Homo sapiens. In other embodiments, the anti-TNFα antibody comprises the amino acid sequence of adalimumab (HUMIRA); infliximab (REMICADE), golimumab (SIMPONI), or an antibody format such as certolizumab pegol (CIMZIA) or with a circulating receptor fusion protein such as etanercept (ENBREL). In some embodiments, the anti-TNFα antibody comprises the amino acid sequence of AMJEVITA, CYLTEZO, HUMIRA or a biosimilar thereof. In some embodiments, the monoclonal antibody comprises the amino acid sequence of full length antibody, an Fab, or an F(ab′)2, of adalimumab (HUMIRA); infliximab (REMICADE), and golimumab (SIMPONI), or antibody formats such as certolizumab pegol (CIMZIA) or with a circulating receptor fusion protein such as etanercept (ENBREL), AMJEVITA, CYLTEZO or a biosimilar thereof. In some embodiments, the anti-TNFα antibody comprises the amino acid sequence of full length antibody, an Fab, or an F(ab′)2, any approved drugs that target TNFα or TNFα pathways (e.g., TNFα receptor).

Such an anti-TNFα antibody, in some embodiments, is a variant of adalimumab. For example, in some embodiments, provided herein is a monoclonal antibody comprising sialylated glycans at NH84 on the variable domain of the heavy chain of the monoclonal antibody. In some embodiments, provided herein is a monoclonal antibody comprising sialylated glycans at NL86 on the variable domain of the light chain of the monoclonal antibody. In some embodiments, provided herein is a monoclonal antibody comprising sialylated glycans at NH84 on the variable domain of the heavy chain and sialylated glycans at NL86 on the variable domain of the light chain of the monoclonal antibody.

In some embodiments, provided herein is a monoclonal antibody comprising one or more of the following structures:

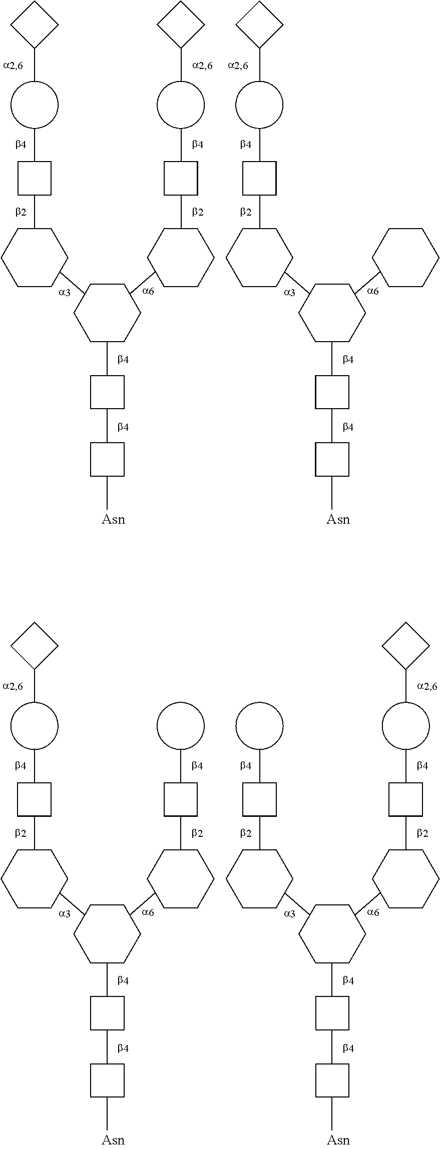

wherein the diamond represents a sialic acid residue, the empty circle represents a galactose residue, the square represents an N-acetylglucosamine residue and the hexagon represents a mannose residue, and wherein the Asn is an Asn of an N-linked glycosylation consensus sequence in a variable domain of the monoclonal antibody.

In some embodiments, provided herein is a monoclonal antibody comprising one or more of the following structures:

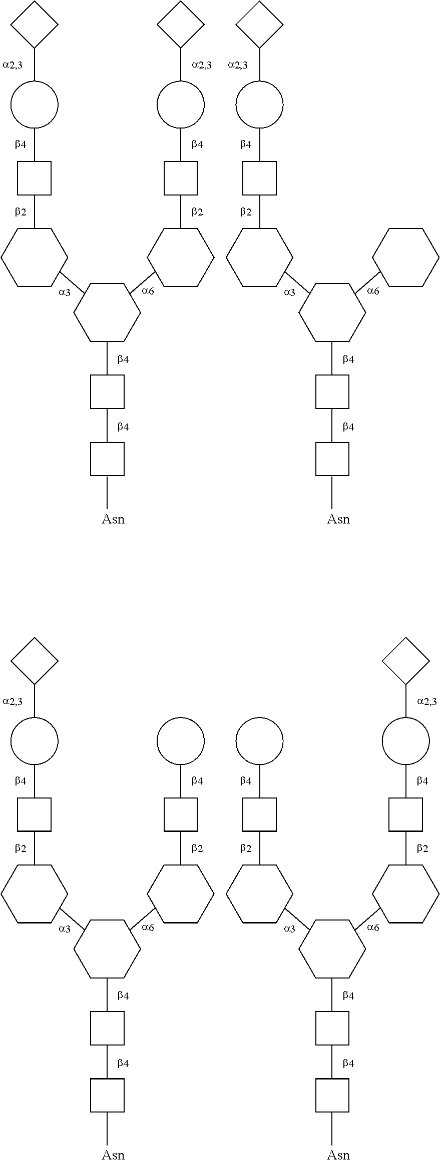

wherein the diamond represents a sialic acid residue, the empty circle represents a galactose residue, the square represents an N-acetylglucosamine residue and the hexagon represents a mannose residue, and wherein the Asn is an Asn of an N-linked glycosylation consensus sequence in a variable domain of the monoclonal antibody.

In some embodiments, provided herein is a monoclonal antibody comprising one or more of the following structures:

| | Oxford/Unifi | Short IgG | Symbol Nomenclature for Glycan |
|---|---|---|---|
| | nomenclature¹ | nomenclature² | structure³ |
| 1 | M3 | Man3 | 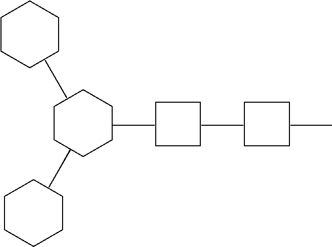 |
| 2 | A1 | G0-N | 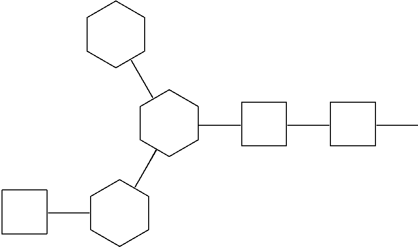 |
| 3 | A1G1 | G1-N | 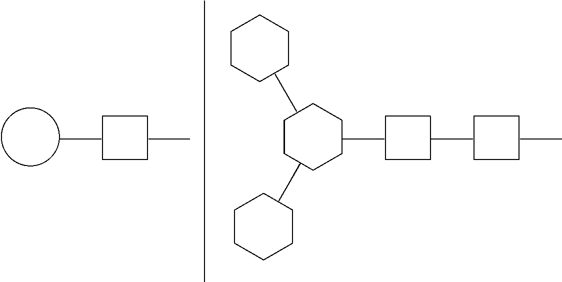 |
| 4 | A1[3]G(4)1 | G1-N | 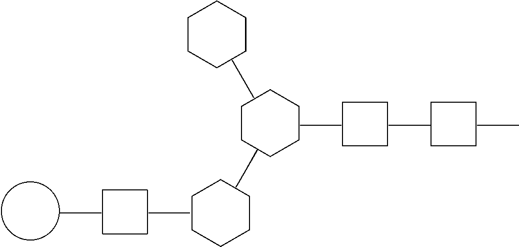 |
| 5 | A2 | G0 | 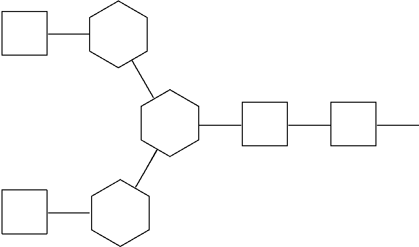 |
| 6 | A2[6]G(4)1 | G1(6′) | 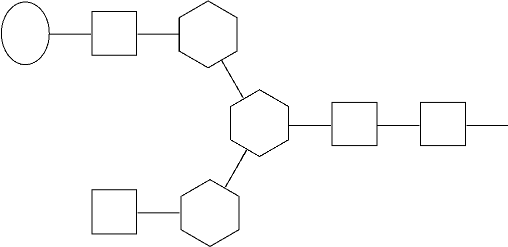 |
| 7 | A2[3]G(4)1 | G1(3′) | 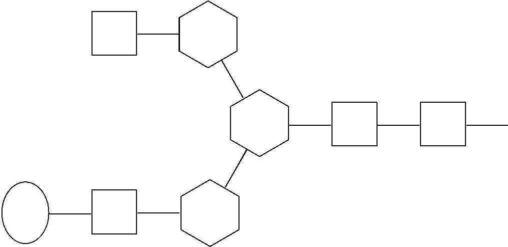 |
| 8 | A2G1 | G1 | 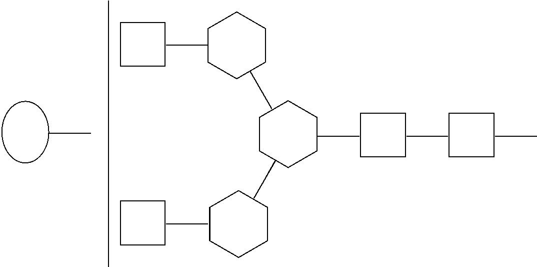 |
| 9 | A1G1S1 | G1S1-N | 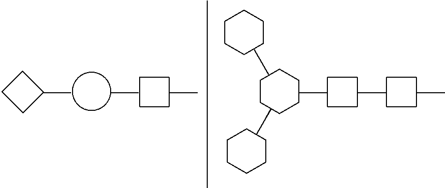 |
| 10 | A1[3]G(4)1S1 | G1S1-N | 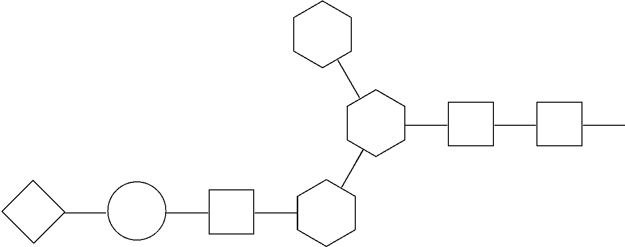 |
| 11 | A2G(4)2 | G2 | 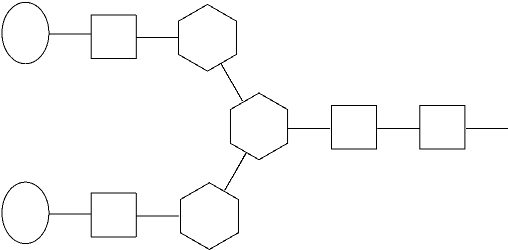 |
| 12 | A2G1S1 | G1S1 | 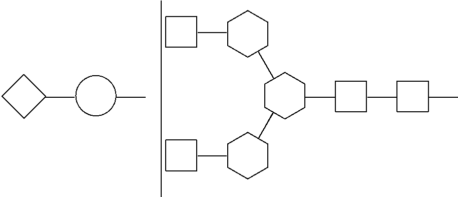 |
| 13 | A2[6]G(4)1S(6)1 | G1S1 | 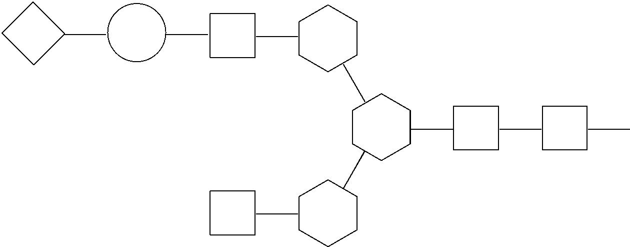 |
| 14 | A2[3]G(4)1S(6)1 | G1S1 | 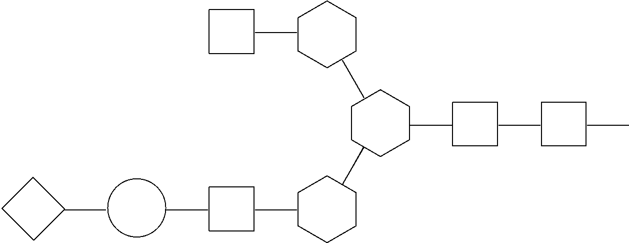 |
| 15 | A2G2S1 | G2S1 | 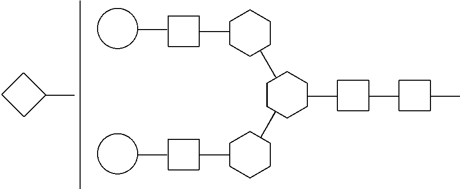 |
| 16 | A2G2S2 | G2S2 | 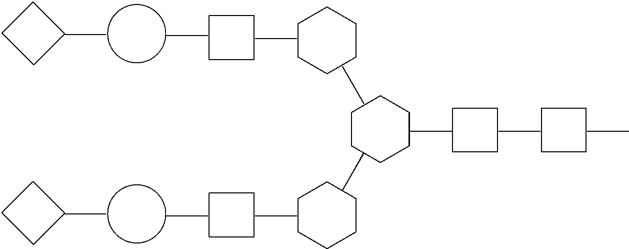 |
| 17 | A2G(4)2S(6,6)2 | G2S2 | 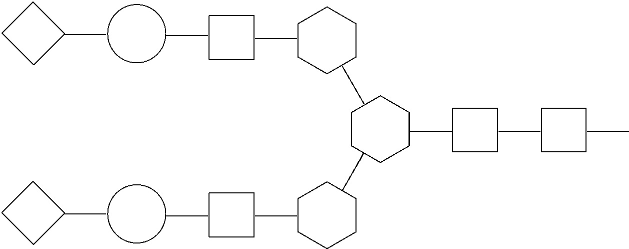 |

¹Mx: number (x) of residues within the oligomannose series;
Ax: number (x) of antennae;
F: core fucose;
Gx: number (x) of galactoses;
B: bisecting GlcNAc;
S: number (x) of sialic acids.
Note:
Linkage information is given in ( ) parentheses if applicable, e.g. F(6)A2 − α1-6 linked fucose. A2G1S1(6) − α2-6 linked sialic acid. Brackets [x] before G indicate which arm of the mannosyl core is galactosylated e.g. [3]G1 indicates that the galactose is on the antenna of the α1-3 mannose.
²This typically with IgG associated naming system indicates the presence of core fucose, the number of galactoses and the presence of biantennary glycans. It is limited in the number of structures and linkages it can describe but is often used for simplicity.
³Hexagon represents mannose (Man), white square is N-acetyl glucosamine (GlcNAc), white circle is galactose (Gal), white diamond is sialic acid, N-acetyl neuraminic acid (Neu5Ac).

wherein the diamond represents a sialic acid residue, the empty circle represents a galactose residue, the square represents an N-acetylglucosamine residue and the hexagon represents a mannose residue, and wherein the reducing end is on Asn of an N-linked glycosylation consensus sequence in the monoclonal antibody.

In some embodiments, a monoclonal antibody provided herein is an anti-TNFα antibody having an antibody-dependent cell mediated cytotoxicity (ADCC) activity that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than that of the same anti-TNFα antibody having a different glycosylation profile.

In some embodiments, a monoclonal antibody provided herein is an anti-TNFα antibody having an ADCC activity against primary inflammatory target cells that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than that of the same anti-TNFα antibody having a different glycosylation profile.

In some embodiments, a monoclonal antibody provided herein is an anti-TNFα antibody having a reduced (lower) immunogenicity that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower than that of the same anti-TNFα antibody having a different glycosylation profile.

In some embodiments, a monoclonal antibody provided herein is an anti-TNF antibody having an increase wound healing M2 macrophages induction activity that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than that of the same anti-TNFα antibody having a different glycosylation profile.

Methods of generating a monoclonal antibody provided herein are well known in the art. Exemplary methods of generating a monoclonal antibody provided herein are described in International Patent Application Publications WO 2017/093291, WO 2019/002512 and WO 2019/234021, which are incorporated herein by reference in their entirety, and are exemplified herein, any one of which can be used to generate a monoclonal antibody provided herein. For example, one of skill in the art will readily appreciate that the nucleic acid sequence of a known protein (e.g., a monoclonal antibody), as well as a newly identified protein (e.g., a monoclonal antibody), can easily be deduced using methods known in the art, and thus it would be well within the capacity of one of skill in the art to introduce a nucleic acid that encodes any monoclonal antibody into a host cell provided herein (e.g., via an expression vector, e.g., a plasmid, e.g., a site specific integration by homologous recombination).

In some embodiments, provided herein is a Leishmania host cell comprising the monoclonal antibody described herein. Such a host cell, in some embodiments, is Leishmania tarentolae. In some embodiments, the host cell is a Leishmania aethiopica cell. In some embodiments, the host cell is part of the Leishmania aethiopica species complex. In some embodiments, the host cell is a Leishmania aristidesi cell. In some embodiments, the host cell is a Leishmania deanei cell. In some embodiments, the host cell is part of the Leishmania donovani species complex. In some embodiments, the host cell is a Leishmania donovani cell. In some embodiments, the host cell is a Leishmania chagasi cell. In some embodiments, the host cell is a Leishmania infantum cell. In some embodiments, the host cell is a Leishmania hertigi cell. In some embodiments, the host cell is part of the Leishmania major species complex. In some embodiments, the host cell is a Leishmania major cell. In some embodiments, the host cell is a Leishmania martiniquensis cell. In some embodiments, the host cell is part of the Leishmania mexicana species complex. In some embodiments, the host cell is a Leishmania mexicana cell. In some embodiments, the host cell is a Leishmania pifanoi cell. In some embodiments, the host cell is part of the Leishmania tropica species complex. In some embodiments, the host cell is a Leishmania tropica cell.

In some embodiments, provided herein is a method for making a monoclonal antibody comprising culturing a Leishmania host cell described herein and isolating the monoclonal antibody.

In some embodiments, provided herein is a monoclonal antibody produced by the method described herein.

Methods of producing a Leishmania host cell and using such host cells to produce a monoclonal antibody are well known in the art. Exemplary methods are described in International Patent Application Publications WO 2017/093291, WO 2019/002512 and WO 2019/234021, which are incorporated herein by reference in their entirety, and are exemplified herein, any one of which can be used to generate a Leishmania host cell and produce a monoclonal antibody provided here. For example, in some embodiments, host cells described herein are cultured using any of the standard culturing techniques known in the art, including, but not limited to, growth in rich media like Brain Heart Infusion, Trypticase Soy Broth or Yeast Extract, all containing 5 μg/ml Hemin. Additionally, incubation can be done at 26° C. in the dark as static or shaking cultures for 2-3 days. In some embodiments, cultures of host cell contain the appropriate selective agents.

TABLE 1
Summary of strains presented in the examples. Some of the strains were produced by several rounds of
transfection building on top of each other.

| Strain | Parental strain | Locus::construct |
|---|---|---|
| St18344 | ATCC ® 30267 ™ | none |
| St18700 | St18344 | Pfr::drMGAT1_IrLdA_drMGAT2_IrLdB_rnMGAT1_IrLdC_hsB4GalT1_ |
| | | IrE_NeuC_IrLiH_cgNal_IrLil_NeuB_IrLmR_d88hST6_IrLiK_NeuA_IrLiL_ |
| | | d88hST6_IrLiM_d88hST6_IrLiE_Sm(pac)_IrLmW_rnMGAT2_IrLdD_ |
| | | hsMGAT1_IrLdF_hsMGAT2_IrLdG_hsB4GalT1_UtrA |
| St19084 | St18700 | Pfr::hsB4GALT1_IrLmH_rnMGAT2_IrLml_gjMGAT1_IrLmJ_agMGAT1_ |
| | | IrLmK_Sm(bsd)_UtrA |
| St19384 | St19084 | Ssu::PolA_sfGNTI |
| | | bl2_IrLmY_drMGAT1B_IrLmZ_rnMGAT2_IrE_mmST6_IrLmS_ |
| | | hCMAS_IrLmT_hCST_IrC_Sm(ble)_UtrA |
| St19739 | St19384 | Ssu::PolA_rnMGAT1_IrLmAC_hsMGAT1_IrLmAD_gjMGAT1_IrLmAE_ |
| | | agMGAT1_IrLmAF_Sm(neo)_UtrA |
| St19788 | St19739 | Ssu::5UTR_AdalimumabLC_IrA_AdalimumabHC(K84N)_IrC_Sm(ntc)_ |
| | | UtrA |
| St19790 | St19739 | Ssu::5UTR_AdalimumabLC(D86N)_IrA_AdalimumabHC(K84N)_IrC_ |
| | | Sm(ntc)_UtrA |
| St19866 | St19790 | OGNT1*L::Sm(hyg) + OGNT2*::Sm(hyg) |
| St19064 | St18700 | Ssu::5UTR_mmST6_IrLmS_CMAS_IrLmT_mmCST- |
| | | GFP_IrLmU_Sm(bsd)_UrtA |
| St19224 | St19064 | Ssu::5UTR_AdalimumabLC_IrA_AdalimumabHC(K84N)_IrC_Sm(ntc)_ |
| | | UtrA |
| St19226 | St19064 | Ssu::5UTR_AdalimumabLC(D86N)_IrA_AdalimumabHC(K84N)_IrC_ |
| | | Sm(ntc)_UtrA |
| St20108 | St19084 | |
| St20208 | St20108 | Ssu::PolA_sfGntI_IrLmY_drMGAT1b_IrLmZ_rnMGAT2_IrE_mmST6_ |
| | | IrLmS_ hCMAS_IrLmT_hCST_IrLmU_Sm(hyg)_UtrA |
| St20224 | St20108 | Ssu::PolA_sfGntI_IrLmY_drMGAT1b_IrLmZ_rnMGAT2_IrE_mmST6_ |
| | | IrLmS_ hCMAS_IrLmT_hCST_IrLmU_hsNGT_IrLdR_Sm(hyg)_UtrA |
| StCGP02824 | St20208 | Ssu::5UTR_AdalimumabLC(D86N)_IrA_AdalimumabHC(K84N)_IrC_ |
| | | Sm(ntc)_UtrA |
| StCGP02826 | St20224 | Ssu::5UTR_AdalimumabLC(D86N)_IrA_AdalimumabHC(K84N)_IrC_ |
| | | Sm(ntc)_UtrA |
| StCGP02944 | StCGP02824 | Ssu::5UTR_AdalimumabLC(D86N)_IrA_AdalimumabHC(K84N)_IrC_ |
| | | Sm(ble)_UtrA |
| StCGP02946 | StCGP02826 | Ssu::5UTR_AdalimumabLC(D86N)_IrA_AdalimumabHC(K84N)_IrC_ |
| | | Sm(ble)_UtrA |

In some embodiments, provided herein is a pharmaceutical composition comprising the monoclonal antibody described herein and a pharmaceutically acceptable carrier.

In some embodiments, provided herein is a method of treating or preventing a disease in a patient comprising administering to the patient a monoclonal antibody described herein or a pharmaceutical composition described herein. In some embodiments, the disease is an inflammatory bowel disease, such as Crohn's disease, pediatric Crohn's disease, ulcerative colitis, microscopic colitis, diverticulosis-associated colitis, collagenous colitis, lymphocytic colitis, or Behcet's disease. In some embodiments, the disease is an inflammatory disease, such as an inflammatory disease selected from rheumatoid arthritis, psoriatic arthritis, ankylosing spondylitis, chronic psoriasis, hidradenitis suppurativa, adult uveitis, pediatric uveitis, plaque psoriasis, and juvenile idiopathic arthritis.

In some embodiments, a method of treating or preventing a disease provided herein include an administration step that comprises intravenous injection, intraperitoneal injection, subcutaneous injection, transdermal injection, or intramuscular injection of a monoclonal antibody described herein or a pharmaceutical composition described herein.

In some embodiments, a method of treating or preventing a disease provided herein requires a lower dose and/or lower administration frequency to achieve the same effect as compared to the same antibody having a different glycosylation profile; and/or can be administered for an extended period of time (at least 1, 2, 3, 4, 5, 6, 7, 8, 9, 10, 11 or at least 12 months, at least 2, 3, 4, 5, 6, 7, 8, 9, or at least 10 years); and/or does not trigger an immune response against the monoclonal antibody in the patient.

In some embodiments, the pharmaceutical compositions described herein can be administered in a single dosage form, for example a single dosage form of a monoclonal antibody described here.

In some embodiments, a method of treating or preventing a disease provided herein requires a lower immunosuppressant co-medication, such as corticosteroids, cyclophosphamide, tacrolimus, azathioprine, cyclosporine, or tofacitinib.

In some embodiments, a suitable dose of a monoclonal antibody described herein is the amount corresponding to the lowest dose effective to produce a therapeutic effect. For example, an effective amount of an anti-TNFα antibody may be an amount that inhibits TNFα activity in a subject suffering from a disease to be detrimental TNFα activity.

In some embodiments, the amount of monoclonal antibody described herein administered to a patient may be not more than the amount listed in the label of a drug product of the same monoclonal antibody having a different glycosylation profile from that of the monoclonal antibody described herein. For example, the amount of adalimumab produced herein administered to a patient may be not more than the amount listed in the label of the HUMIRA drug product. In some embodiments, the frequency of administration of a monoclonal antibody described herein administered to a patient may be not more than the frequency list in the label of a drug product of the same monoclonal antibody having a different glycosylation profile from that of the monoclonal antibody described herein. For example, the frequency of administration of adalimumab produced herein administered to a patient may be not more than the frequency listed in the label of HUMIRA drug product.

In some embodiments, the accumulated amount of a monoclonal antibody described herein administered to a patient over a period of time may be not more than the accumulated amount indicated in the label of a drug product of the same monoclonal antibody having different glycosylation profile from that of the monoclonal antibody described herein. In some embodiments, the reduced accumulated amount could be administered in reduced doses on a reduced frequency. In some embodiments, the reduced accumulated amount could be administered in one or more doses that are the same or higher than the dose in the label on a reduced frequency. In some embodiments, the reduced accumulated amount could be administered in one or more reduced doses on a frequency that is the same or higher than the frequency in the label. In some embodiments, the reduced accumulated amount could be administered over a shorter period of time than the period of time for the drug product to achieve the same level of effect in treatment or prevention.

In some embodiments, the amount of the monoclonal antibody described herein in a single dose administered to a patient can be from about 1 to 150 mg, about 5 to 145 mg, about 10 to 140 mg, about 15 to 135 mg, about 20 to 130 mg, about 25 to 125 mg, about 30 to 120 mg, about 35 to 115 mg, about 40 to 110 mg, about 45 to 105 mg, about 50 to 100 mg, about 55 to 95 mg, about 60 to 90 mg, about 65 to 5 mg, about 70 to 80 mg, or about 75 mg. In some embodiments, the amount of monoclonal antibody described herein in a single dose administered to a patient can be from about 5 to about 80 mg. In some embodiments, the amount of monoclonal antibody described herein in a single dose administered to a patient can be from about 25 to about 50 mg. In some embodiments, the amount of a monoclonal antibody described herein in a single dose administered to a patient can from about 15 mg to about 35 mg.

In some embodiments, the amount of a monoclonal antibody described herein in a single dose administered to a patient can be no more than 40 mg, for example 40 mg, 35 mg, 30 mg, 25 mg, 20 mg, 18 mg, 15 mg, 12 mg, 10 mg, 7 mg, 5 mg, and 2 mg. In some embodiments, the amount of a monoclonal antibody described herein in a single dose administered to a patient can be no more than 80 mg, for example 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a monoclonal antibody described herein in a single dose administered to a patient can be no more than 160 mg, for example 150 mg, 140 mg, 130 mg, 120 mg, 110 mg, 100 mg, 90 mg, 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a monoclonal antibody described herein in a single dose administered to a patient can be equal to or more than 160 mg, for example 170 mg, 180 mg, 200 mg, 250 mg, and 300 mg.

In some embodiments, a monoclonal antibody of the disclosure can be administered on a frequency that is every other week, namely every 14 days. In some embodiments, a monoclonal antibody of the disclosure can be administered on a frequency that is lower than every 14 days, for example, every half a month, every 21 days, monthly, every 8 weeks, bimonthly, every 12 weeks, every 3 months, every 4 months, every 5 months, or every 6 months. In some embodiments, a monoclonal antibody of the disclosure can be administered on a frequency that is the same or higher than every 14 days, for example, every 14 days, every 10 days, every 7 days, every 5 days, every other day, or daily.

In some embodiments, the administration of a monoclonal antibody of the disclosure can comprise an induction dose that is higher than the following doses, for example the following maintenance doses. In some embodiments, the administration of a monoclonal antibody of the disclosure can comprise a second dose that is lower than the induction dose and higher than the following maintenance doses. In some embodiments, the administration of a monoclonal antibody of the disclosure can comprise the same amount of the monoclonal antibody in all the doses throughout the treatment period.

In some embodiments, a method of treating or preventing a disease provided herein includes the disease being rheumatoid arthritis, psoriatic arthritis, or ankylosing spondylitis, and wherein the method comprises administering to the patient less than or equal to 40 mg of an anti-TNFα antibody described herein on an administration frequency less than or equal to every other week.

In some embodiments, a method of treating or preventing a disease provided herein includes the disease being Crohn's disease or ulcerative colitis, and wherein the method comprises administering to the patient less than or equal to 160 mg of an anti-TNFα antibody described herein on day 1, less than or equal to 80 mg of an anti-TNFα antibody described herein on day 15, and less than or equal to 40 mg of an anti-TNFα antibody described herein on an administration frequency less than or equal to every other week starting on day 29.

In some embodiments, a method of treating or preventing a disease provided herein includes the disease being pediatric Crohn's disease, and wherein the method comprises administering to the patient: less than or equal to 80 mg of an anti-TNFα antibody described herein on day 1, less than or equal to 40 mg of an anti-TNFα antibody described herein on day 15, and less than or equal to 20 mg of an anti-TNFα antibody described herein on an administration frequency less than or equal to every other week starting on day 29 in a patient having a body weight between 17 kg and 40 kg, or less than or equal to 160 mg of an anti-TNFα antibody described herein on day 1, less than or equal to 80 mg of an anti-TNFα antibody described herein on day 15, and less than or equal to 40 mg of an anti-TNFα antibody described herein on an administration frequency less than or equal to every other week starting on day 29 in a patient having a body weight equal to or higher than 40 kg.

In some embodiments, a method of treating or preventing a disease provided herein includes the disease being juvenile idiopathic arthritis or pediatric uveitis, and wherein the method comprises administering to the patient: less than or equal to 10 mg of an anti-TNFα antibody described herein on an administration frequency less than or equal to every other week in a patient having a body weight between 10 kg and 15 kg, less than or equal to 20 mg of an anti-TNFα antibody described herein on an administration frequency less than or equal to every other week in a patient having a body weight between 15 kg and 30 kg, or less than or equal to 40 mg of an anti-TNFα antibody described herein on an administration frequency less than or equal to every other week in a patient having a body weight equal to or higher than 30 kg.

In some embodiments, a method of treating or preventing a disease provided herein includes the disease being plaque psoriasis or adult uveitis, and wherein the method comprises administering to the patient less than or equal to 80 mg of an anti-TNFα antibody described herein on day 1, and less than or equal to 40 mg on an administration frequency less than or equal to every other week starting on day 8.

In some embodiments, a method of treating or preventing a disease provided herein includes the disease being hidradenitis suppurativa, and wherein the method comprises administering to the patient: less than or equal to 80 mg of an anti-TNFα antibody described herein on day 1, and less than or equal to 40 mg of an anti-TNFα antibody described herein on an administration frequency less than or equal to every other week starting on day 8 in an adolescent patient who are 12 years and older having a body weight between 30 kg and 60 kg, or less than or equal to 160 mg of an anti-TNFα antibody described herein on day 1, and less than or equal to 80 mg of an anti-TNFα antibody described herein on day 15, and less than or equal to 40 mg of an anti-TNFα antibody described herein on an administration frequency less than or equal to every week starting on day 29 in an adolescent patient who are 12 years and older having a body weight equal to or higher than 60 kg or an adult patient.

In some embodiments, provided herein is a single dosage form of a monoclonal antibody described herein. In some embodiments, the single dosage form consists of about 2 mg, about 5 mg, about 7 mg, about 10 mg, about 12 mg, about 15 mg, about 18 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg of a monoclonal antibody (e.g., an anti-TNFα antibody) described herein. Such single dosage form can, in some embodiments, be a prefilled syringe, an injection pen, a vial, a tablet, or a capsule. Additionally, such single dosage form can comprise a monoclonal antibody (e.g., an anti-TNFα antibody) described herein in a lyophilized form or in a liquid solution.

It is understood that modifications which do not substantially affect the activity of the various embodiments of this invention are also provided within the definition of the invention provided herein. Accordingly, the following examples are intended to illustrate but not limit the present invention.

### EXAMPLE I

### Production of Glycoengineered Adalimumab Variants

Glycoengineered adalimumab variants (A-84S or A8486S) were generated using different CGP cell lines using standard protocols. The CGP cell lines contain glycoengineering elements such as those described in the International Patent Application Publications WO 2019/002512 and WO 2019/234021, which are incorporated herein by reference. Cell lines St19224 and St19226 contain specifically open reading frames for drMGAT1, drMGAT2, rnMGAT1, hsB4GalT1, NeuC, cgNal, NeuB, hsST6, rnMGAT2, hsMGAT1, hsMGAT2, and NeuA in the Pfr locus, mmST6, CMAS, and CST in the ssu ribosomal DNA locus, and contain either adalimumab K84N (heavy chain) in St19224 or K84N (heavy chain)_D86N (light chain) in St19226. Cell lines St19788 and S19790 both contain open reading frames in the Pfr locus for drMGAT1, drMGAT2, rnMGAT1, hsB4GalT1, NeuC, CgNal, NeuB, hsST6, NeuA, rnMGAT2, hsMGAT1, hsMGAT2, and hsB4GalT1, in a second Pfr locus hsB4GALT1, rnMGAT2, gjMGAT1, and agMGAT1, and in the ssu-Poll ribosomal DNA locus sfGNTI, drMGAT1B, rnMGAT2 , mmST6, CMAS, hsCST, rnMGAT1, hsMGAT1, gjMGAT1, and agMGAT1. St19788 encodes adalimumab K84N (heavy chain) and St19790 encodes K84N (heavy chain)_D86N (light chain). Such glycoengineered adalimumab variants were analyzed as described in the Examples that follow. Table 1 provides the quality control parameters of these glycoengineered adalimumab variants.

TABLE 1
Quality control (QC) parameters of glycoengineered adalimumab (A-84S or A-8486S) derived from different CGP cell lines

| | | | | A-84S | A-8486S | A-84-S | A-8486-S |
|---|---|---|---|---|---|---|---|
| | | | A-84-S (IVGE) | st19224 | st19226 | St19788 | St19790 |
| Parameter | Assay | Definition | 16-1088 | 16-1422 | 16-1423 | 16-1556 | 16-1553 |
| Aggregates | SEC | <5% high | monomeric: | monomeric: | monomeric: | monomeric: | monomeric: |
| | | molecular | 96.3% | 91.9% | 90.3% | 95.1% | 93.1% |
| | | weight | HMW: 2.2% | HMW: 0.9% | HMW: 1.0% | HMW: 0.9% | HMW: 1.5% |
| | | (HMW) | shoulder: | shoulder: | shoulder: | shoulder: | shoulder: |
| | | | 1.0% | 6.1% | 6.8% | 4.0% | 5.2% |
| | | | LMW: 0.0% | LMW: 1.9% | LMW: 2.0% | LMW: 0.0% | LMW: 0.2% |
| Stability - 3x F/T | SEC | <5% HMW | n.d. | monomeric: | monomeric: | n.d. | n.d. |
| | | | | 92.1% | 90.4% | | |
| | | | | HMW: 1.0% | HMW: 1.2% | | |
| Stability - 37° C.; | SEC | comparable | n.d. | monomeric: | monomeric: | n.d. | n.d. |
| 10 d | | to HUMIRA | | 90.2% | 89.0% | | |
| | | | | HMW: 1.4% | HMW: 1.0% | | |
| Purity/ | PAGE Non- | Main | 97.2% | 93.7% | 93.0% | 96.1% | 91.1% |
| degradation | reducing | band ≥90% | | | | | |
| Purity/ | PAGE Reducing | Sum of heavy | 97.1% | 98.3% | 98.8% | 100% | 98.0% |
| degradation | | and light | | | | | |
| | | chain ≥95% | | | | | |
| Purity - 108 kDa | RP-HPLC | <5% | n.d. | 4.4% | 5.4% | 1.6% | 3.5% |
| mAb fragment | | | | | | | |
| N-glycan - Fab | PC labelling of PNGaseF released | | | | | | |
| | glycans from Fab site | | | | | | |
| «Sialylation» | Total Sialylated | | 93% | 84% | 73% | 93% | 93% |
| «G2S1» | A2G(4)2S(6)1 | | 1.4% | 30% | 27% | 30% | 27% |
| «G2S2» | A2G(4)2S(6, 6)2 | | 53% | 32% | 28% | 50% | 50% |
| N-glycan - Fc | PC labelling of PNGaseF released | | | | | | |
| | glycans from Fc site | | | | | | |
| «Fucosylation» | Total fucosylated | | 0% | 0% | 0% | 0% | 0% |
| «Man3» | M3 | | 42% | 56% | 56% | 13% | 13% |
| «G0» | A2 | | 43% | 41% | 39% | 57% | 54% |
| Endotoxin | LAL | <1 EU/mg | 0.116 EU/mg | 0.11 EU/mg | 0.24 EU/mg | 0.7 EU/mg | 0.7 EU/mg |
| Binding to hTNFα | Binding ELISA | Normalized | n.d. | n.d. | n.d. | 121% | 116% |
| | | EC50 50- | | | | | |
| | | 200% vs | | | | | |
| | | HUMIRA | | | | | |
| Binding to hTNFα | IC formation by | Yes | n.d. | n.d. | n.d. | Yes | Yes |
| | SE-HPLC | | | | | | |

### EXAMPLE II

### Fab Glycosylation after Expression in CGP Cell Line St19788

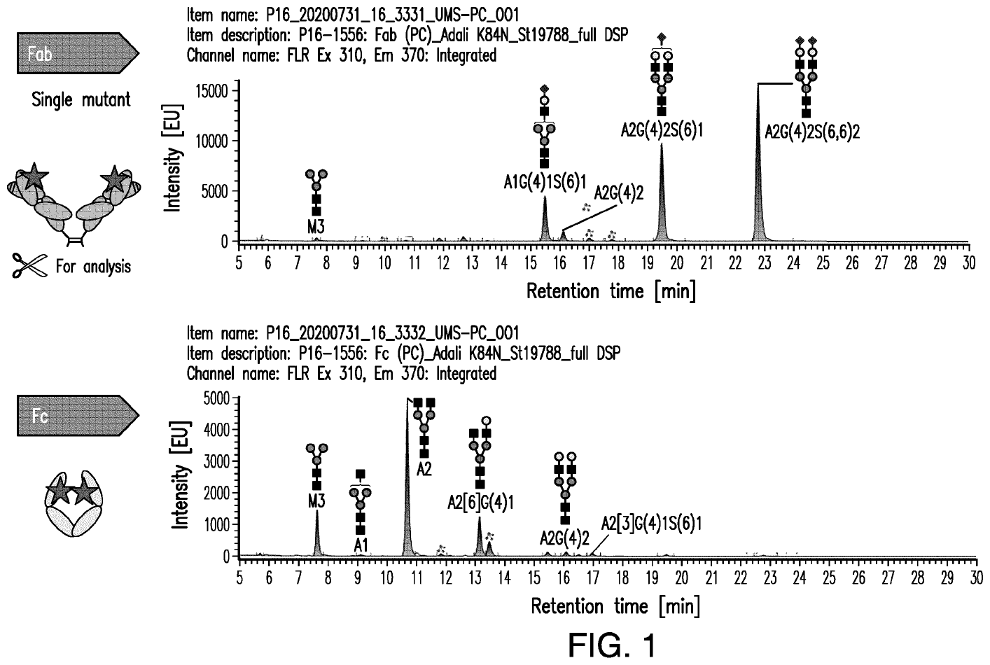

Adalimumab K84N (A-84S) was purified from cell culture supernatant with Protein A, CaptoAdhere and CaptoSP, and formulated in PBS buffer pH6.4. For glycan analysis, the monoclonal antibody was cleaved with IdeZ to F(ab′)2 and Fc/2 (left panel, schematic representation), separated on SDS PAGE and bands were excised and enzymatic release of N-glycans from the monoclonal antibody was performed using PNGase F. Following release, glycans were directly labeled with procainamide (PC). PC-labeled N-glycans were analyzed by HILIC-UPLC-MS with fluorescence detection coupled to a mass spectrometer. Glycans were separated using an Acquity BEH Amide column. Data processing and analysis was performed using Unifi. Glucose units were assigned on the retention times of a procainamide-labeled dextran ladder. Glycan structures were assigned based on their m/z values and their retention times. Glycan forms and relative percentages were calculated based on peak areas. As shown in FIG. 1, N-glycans of the Fab glycosylation site of adalimumab K84N present with high abundance of alpha 2,6 biantennary sialylation and N-glycans of conserved N- glycosite 297 in the Fc part after expression in St19788.

### EXAMPLE III

Fab Glycosylation after Expression in CGP Cell Line St19790

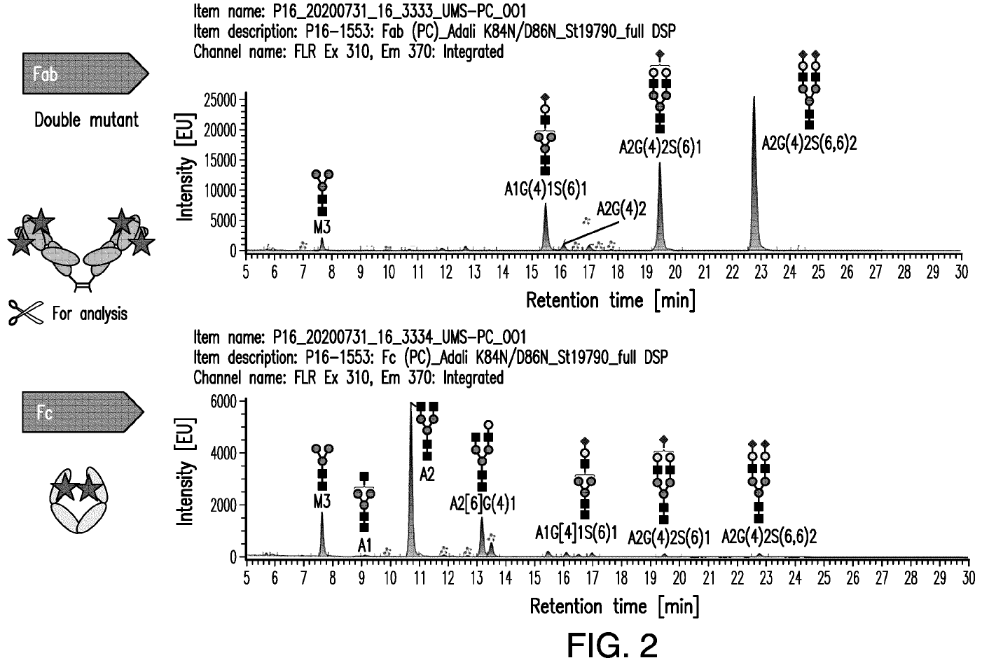

Adalimumab K84N-D86N (A-8486S) was purified from cell culture supernatant with Protein A, CaptoAdhere and CaptoSP, and formulated in PBS buffer pH6.4. For glycan analysis the monoclonal antibody was cleaved with IdeZ to F(ab′)2 and Fc/2 (left panel, schematic representation), separated on SDS PAGE and bands were excised and enzymatic release of N-glycans from the monoclonal antibody was performed using PNGase F. Following release, glycans were directly labeled with procainamide (PC). PC-labeled N-glycans were analyzed by HILIC-UPLC-MS with fluorescence detection coupled to a mass spectrometer. Glycans were separated using an Acquity BEH Amide column. Data processing and analysis was performed using Unifi. Glucose units were assigned on the retention times of a procainamide-labeled dextran ladder. Glycan structures were assigned based on their m/z values and their retention times. Glycan forms and relative percentages were calculated based on peak areas. As shown in FIG. 2, N-glycans of the Fab glycosylation site of adalimumab K84N-D86N present with high abundance of alpha 2,6 biantennary sialylation and N-glycans of conserved N-glycosite 297 in the Fc part after expression in St19790.

### EXAMPLE IV

### N-Glycan Abundance

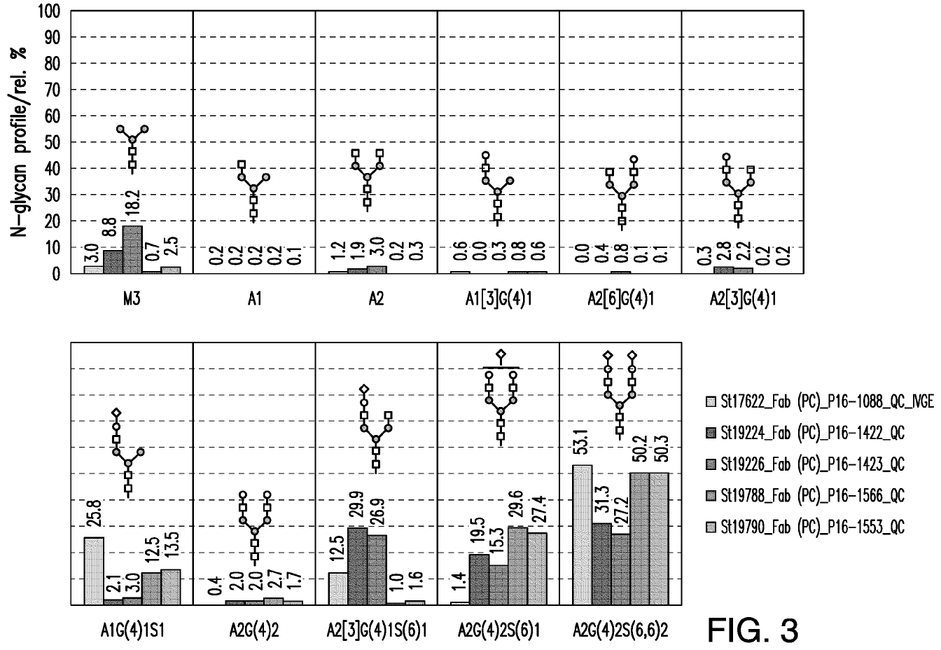

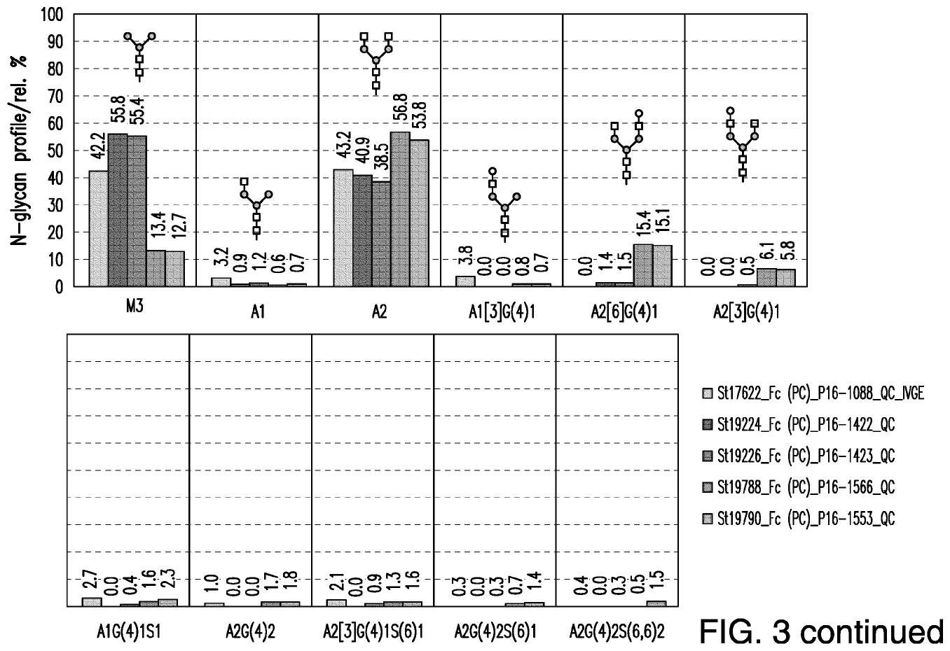

The relative abundance of N-glycans on antibodies expressed in different CGP cell line backgrounds was determined using standard protocols. The top panel of FIG. 3 shows the relative quantification of glycoforms released from the Fab glycosites (K84N or K84N-D86N from A-84S or A-8486S, respectively). The bottom panel of FIG. 3 show the conserved Fc glycosite, after separation by IdeZ cleavage. Nomenclature and glycan annotation is found in Table 2.

TABLE 2
N-glycans nomenclature and abbreviations

| | Oxford/Unifi | Short IgG | Symbol Nomenclature for Glycan |
|---|---|---|---|
| | nomenclature¹ | nomenclature² | structure³ |
| 1 | M3 | Man3 | 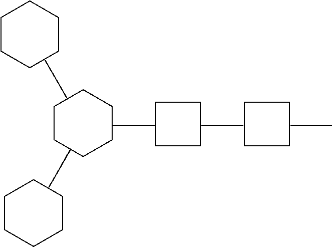 |
| 2 | A1 | G0-N | 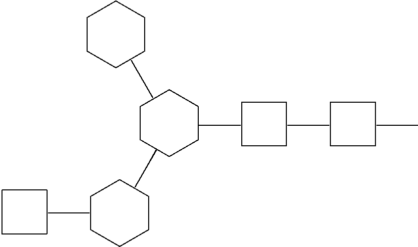 |
| 3 | A1G1 | G1-N | 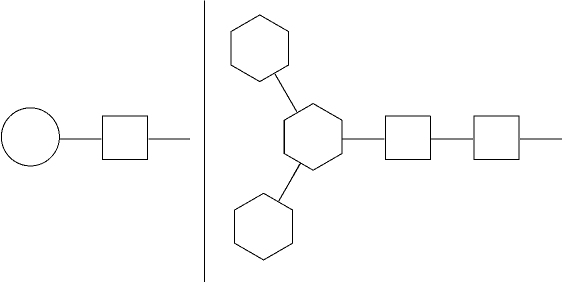 |
| 4 | A1[3]G(4)1 | G1-N | 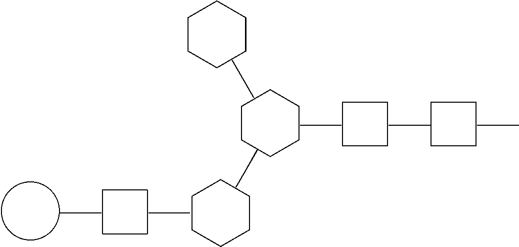 |
| 5 | A2 | G0 | 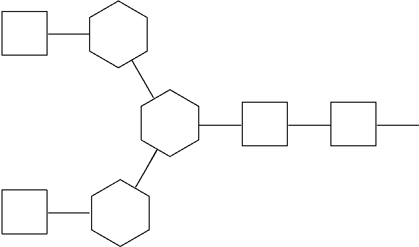 |
| 6 | A2[6]G(4)1 | G1(6′) | 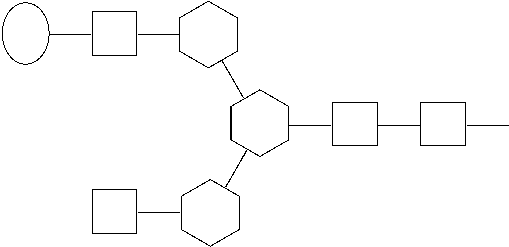 |
| 7 | A2[3]G(4)1 | G1(3′) | 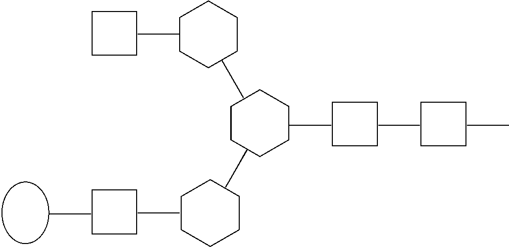 |
| 8 | A2G1 | G1 | 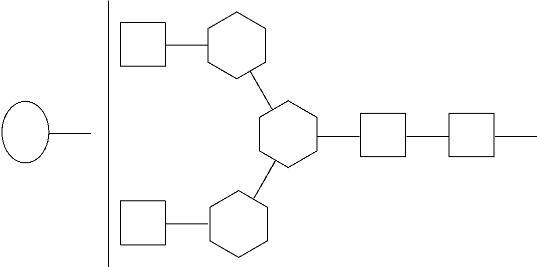 |
| 9 | A1G1S1 | G1S1-N | 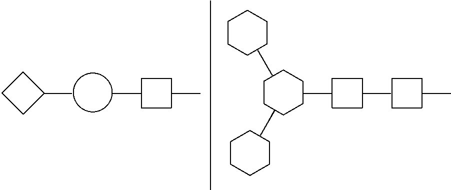 |
| 10 | A1[3]G(4)1S1 | G1S1-N | 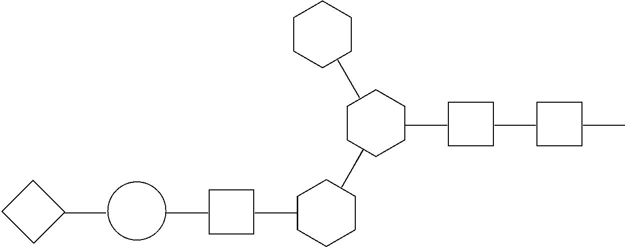 |
| 11 | A2G(4)2 | G2 | 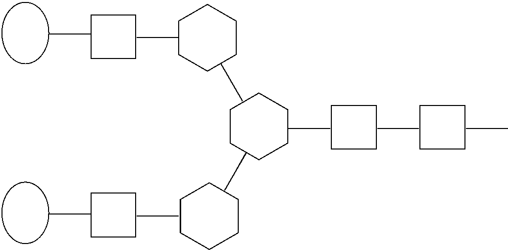 |
| 12 | A2G1S1 | G1S1 | 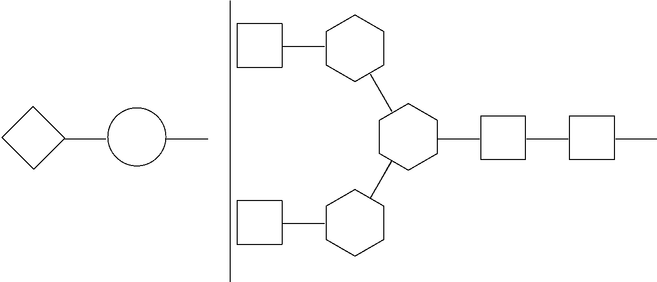 |
| 13 | A2[6]G(4)1S(6)1 | G1S1 | 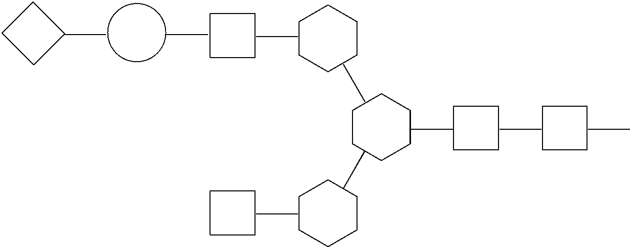 |
| 14 | A2[3]G(4)1S(6)1 | G1S1 | 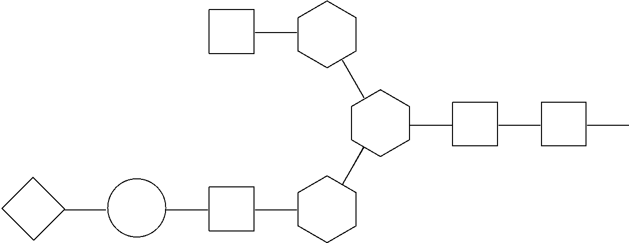 |
| 15 | A2G2S1 | G2S1 | 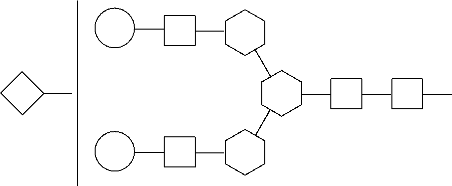 |
| 16 | A2G2S2 | G2S2 | 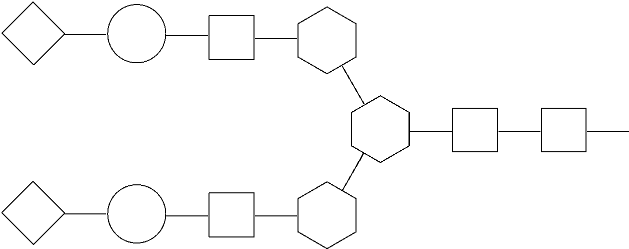 |
| 17 | A2G(4)2S(6,6)2 | G2S2 | 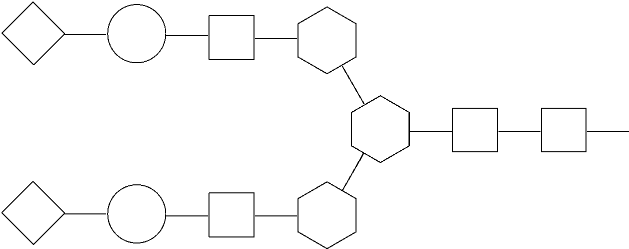 |

¹Mx: number (x) of residues within the oligomannose series;
Ax: number (x) of antennae;
F: core fucose;
Gx: number (x) of galactoses;
B: bisecting GlcNAc;
S: number (x) of sialic acids.
Note:
Linkage information is given in ( ) parentheses if applicable, e.g. F(6)A2 − α1-6 linked fucose. A2G1S1(6) − α2-6 linked sialic acid. Brackets [x] before G indicate which arm of the mannosyl core is galactosylated e.g. [3]G1 indicates that the galactose is on the antenna of the α1-3 mannose.
²This typically with IgG associated naming system indicates the presence of core fucose, the number of galactoses and the presence of biantennary glycans. It is limited in the number of structures and linkages it can describe but is often used for simplicity.
³Hexagon represents mannose (Man), white square is N-acetyl glucosamine (GlcNAc), white circle is galactose (Gal), white diamond is sialic acid, N-acetyl neuraminic acid (Neu5Ac).

### EXAMPLE V

### Fab Sialylated Glycans Access and Recognition

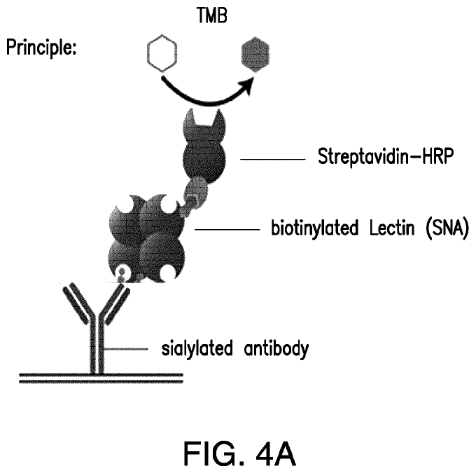

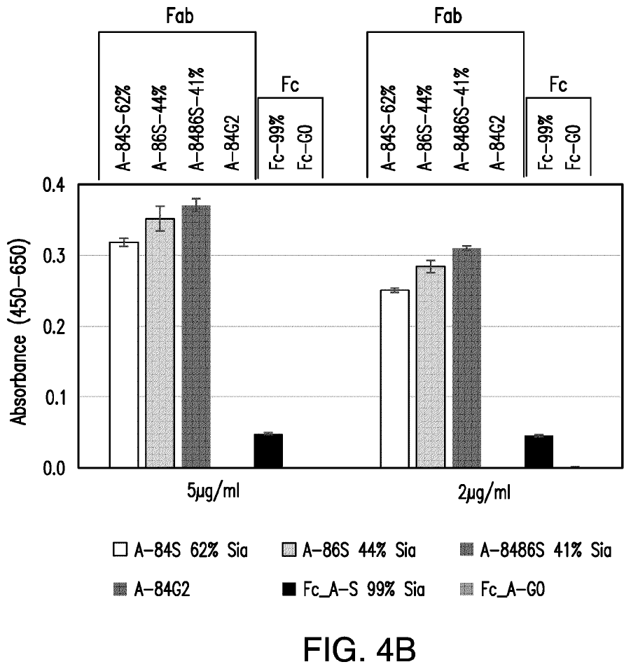

As shown in FIGS. 4A and 4B, Fab sialylated glycans are accessible and recognized by SNA lectin, in contrast to Fc sialylated glycans. An ELISA assay was performed as shown in FIG. 4A. FIG. 4B shows the average±SD of duplicates measurements of absorbance values for each conditions. Within FIG. 4B, the following can be seen: Fab indicates that the antibody coated contained a N-glycan in the Fab region as well as in the Fc (canonical N-297 position); Fc indicates that the antibody coated contained a N-glycan only in the Fc (canonical N-297 position); A-84S indicates an adalimumab variant with a sialylated N-glycan at position 84 of the variable heavy chain; A-86S indicates an adalimumab variant with a sialylated N-glycan at position 86 of the variable light chain; A-84865 indicates an adalimumab variant with sialylated N-glycan at position 86 of the variable light chain and position 84 of the variable heavy chain; A-84G2 indicates an adalimumab variant with a galactosylated N-glycan at position 84 of the variable heavy chain; Fc_A-S indicates an adalimumab in which the Fc N-glycan (position N-297) is sialylated, this variant does not contain Fab N-glycans; and Fc_A-G0 indicates an adalimumab in which the Fc N-glycan (position N-297) is a G0 , this variant does not contain Fab N-glycans. This data shows that only variants with sialylated N-glycan in the Fab portion are recognized by the SNA lectin.

This observation that only Fab sialylated N-glycans are accessible to be recognized by lectins was extended on Siglec-2 (CD22) the principal sialic acid binding lectin expressed on B lymphocytes. CHO cells stably expressing human CD22 (CHO-hCD22) (Chen et al. 2010). and parental CHO-K1 cells were grown in Ham's F12 media supplemented with stable glutamine, 10% FBS, and Penicillin-Streptomycin, as well as 0.5 mg/ml Hygromycin (CHO-hCD22 only). Cells were grown at 37° C., 5% CO2, in a humidified incubator. Cells were grown on non-tissue culture treated plates before the binding experiment. Cells were removed from the incubator, washed once with phosphate-buffered saline (PBS) supplemented to 5 mM EDTA, and then detached with PBS—5 mM EDTA and vigorous pipetting. Cells were centrifuged at 150 g and then washed two times by resuspension in PBS and centrifugation at 150 g. Cells were stained with Zombie Green dye for 10 min at room temperature, and washed once in PBS supplemented with 0.5% BSA. Cells were resuspended in PBS—0.5% BSA—0.1% sodium azide. Around 4×10⁵ cells were used per assay point in a final volume of 50 μl. Antibody-TNF immune complexes (IC) were preformed as follows: Human TNF-alpha (Peprotech AF-300-01A) reconstituted at 1 mg/ml following manufacturer's instruction, and antibodies (Humira or adalimumab glycovariants, stock at 1 mg/ml) were mixed. Three (3) μl of TNF stock+14 μl antibody+10 μl PBS pH 7.4 were gently mixed by pipetting and incubated 30 minutes at room temperature. The solution was considered to be IC at 0.5 mg/ml. The IC solution was used within the next 30 min for staining. Antibody-TNF ICs were added on CHO cells to a final concentration of 10 μg/ml and incubated 45 min on ice. Cells were washed once with 800 μl PBS—0.5% BSA—0.1% Sodium azide, and then stained with APC anti-human IgG Fc antibody before analysis on an BD Accuri C6 flow cytometer. Data was analyzed using FCS Express 6 (De Novo Software). Median fluorescence intensities (MFI) were determined, and the specific CHO-CD22 staining intensity was calculated as the MFI CHO-hCD22—MFI CHO-K1.

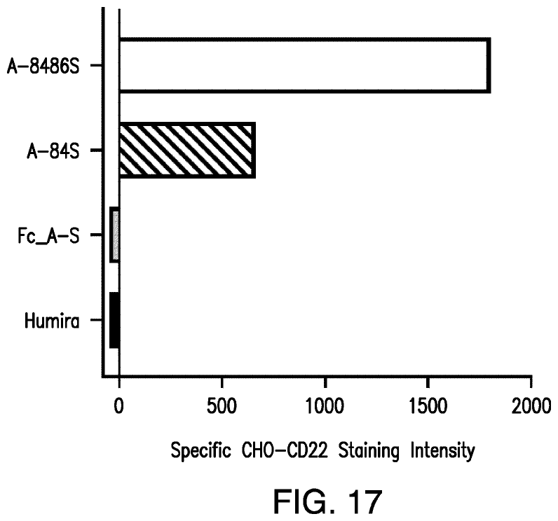

FIG. 17 shows that only A-84S and A-84865 variants, but not the variant with sialylated N-glycan in the Fc fragment only (Fc_A-S) could bind specifically to the CHO-hCD22 cells, indicating binding to CD22. As a control, Humira which displays non sialylated Fc N-glycan did not bind to CHO-CD22 either. Interestingly, the A-8486S variant demonstrated a significantly higher binding to CD22 than the A-84S variant which has half the sialic load, indicating that the amount of sialic acid displayed on the Fab portion is important to drive a better recognition by CD22. Within FIG. 17 the following can be seen: The X-axis of the graph shows the specific CHO-CD22 staining intensity for indicated antibodies. A-84S indicates an adalimumab variant with a sialylated N-glycan at position 84 of the variable heavy chain; A-86S indicates an adalimumab variant with a sialylated N-glycan at position 86 of the variable light chain; A-8486S indicates an adalimumab variant with sialylated N-glycan at position 86 of the variable light chain and position 84 of the variable heavy chain; Fc_A-S indicates an adalimumab in which the Fc N-glycan (position N-297) is sialylated, this variant does not contain Fab N-glycans.

CD22 is an important sialic-specific receptor which has been shown to inhibit B lymphocyte activation and drive B lymphocyte apoptosis. These data therefore support the hypothesis that a Fab sialylated format of adalimumab, such as A-8486S, will engage efficiently CD22 on B lymphocytes and modulate B lymphocyte activation, notably activation derived from the B cell receptor signaling. B lymphocytes bearing a BCR specific for a drug antibody such as adalimumab will therefore be less activated, or enter in apoptosis if CD22 is co-engaged by a Fab sialylated version of adalimumab such as A-8486S. This will lead to lower immunogenicity of the A-8486S antibody, as compared to the parental adalimumab antibody which does not engage CD22.

### EXAMPLE VI

### Immunogenicity of Fab-Sialylated Versus Fc-Sialylated

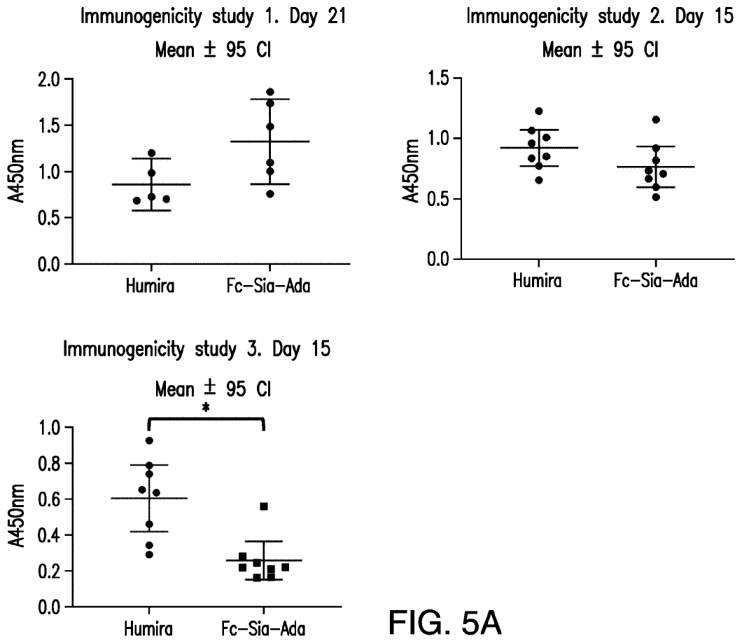

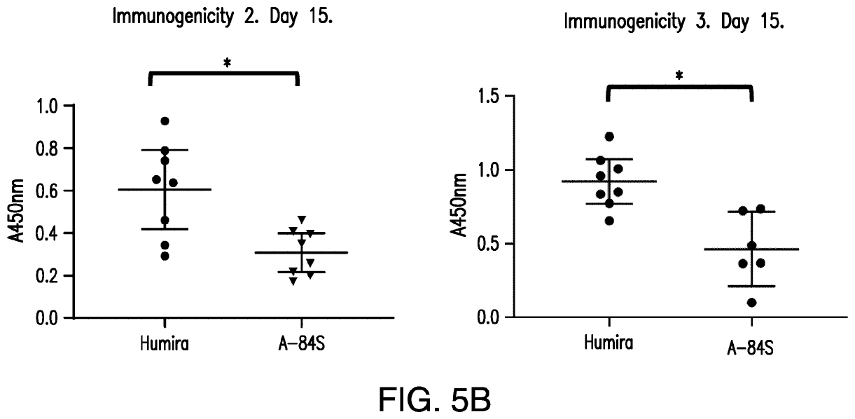

C57BL/6 mice were injected intravenously with 5 mg/kg HUMIRA or glycosylated variants of HUMIRA. Anti-adalimumab antibodies were measured at indicated days in serum of animals using standard ELISA method. The graphs of FIGS. 5A and 5B show the individual animal absorbance levels, mean±95% confidence intervals. The stars in FIGS. 5A and 5B indicate statistical significance (p<0.05) according a non-parametric Kruskal-Wallis test with Dunn's correction. FIG. 5A show results from experiments with adalimumab variant with sialylated N-glycan in the Fc fragment only (Fc-Sia-Ada). FIG. 5B show results from experiments with an adalimumab variant with a sialylated N-glycan at position 84 of the variable heavy chain (A-84S). HUMIRA is adalimumab. This data shows that adalimumab variant A-84S with a sialylated N-glycan in the Fab part displays reduced immunogenicity compared to adalimumab (HUMIRA), whereas Fc-sialylated adalimumab does not consistently show reduced immunogenicity.

### EXAMPLE VII

### M2 Macrophage Induction

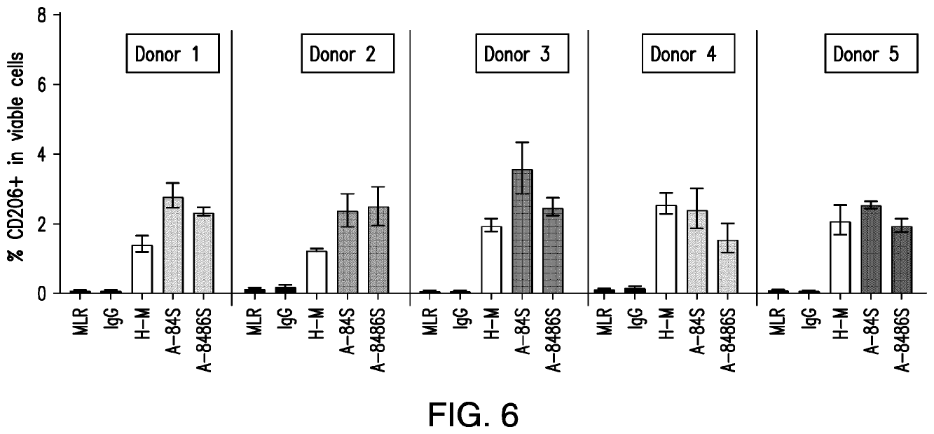

Mixed lymphocyte reaction were induced by mixing human PBMCs from 2 different human donors. A total of 5 different MLR pairs was performed. Antibodies were added to the culture at 0.2 μg/ml. After 7 days of MLR, the samples were stained for CD14, CD206 and CD163 and acquired on a flow cytometer. CD14 is a myeloid marker and CD206 and CD163 are markers of M2 macrophage phenotype. The % of M2 macrophages in the MLR was determined by gating the CD14+CD206+ population, in viable cells. The graphs of FIG. 6 show the mean of quadruplicates values±SEM of % of M2 macrophages at the end of MLR cultures. Within FIG. 6, the following can be seen: MLR is the condition without antibody addition; IgG is addition of an IgG1 control antibody; A-84S indicates an adalimumab variant with a sialylated N-glycan at position 84 of the variable heavy chain; and A-8486S indicates an adalimumab variant with sialylated N-glycan at position 86 of the variable light chain and position 84 of the variable heavy chain. This data shows that Fab-sialylated adalimumab variants show a trend for increase formation of M2 macrophages in 3 out of 5 donor pairs tested. M2 macrophages have an important function in mucosal healing in IBD patients.

### EXAMPLE VIII

### Antibody-Dependent Cellular Cytotoxicity (ADCC)

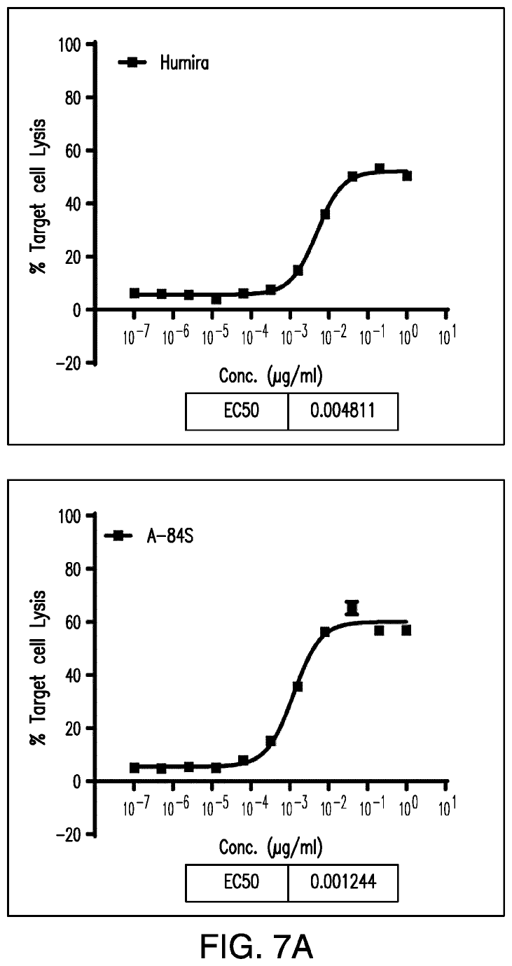

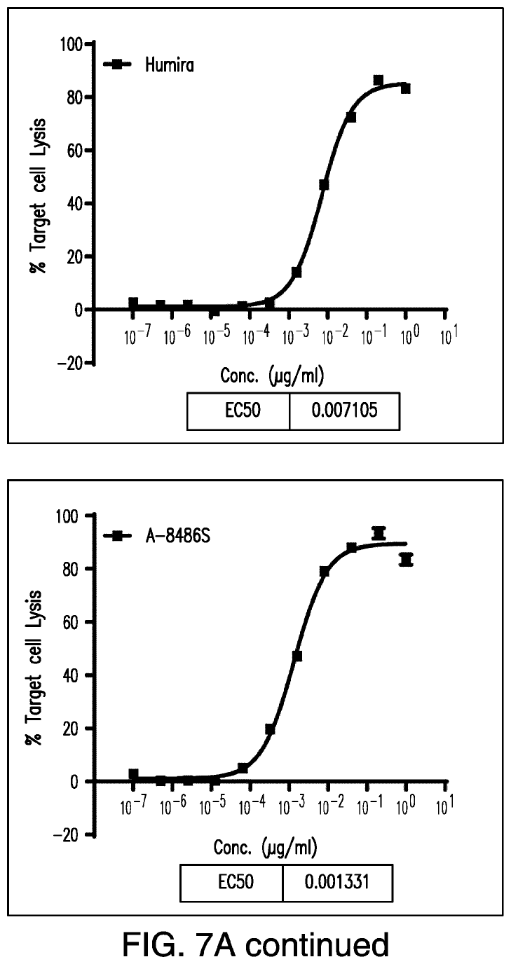

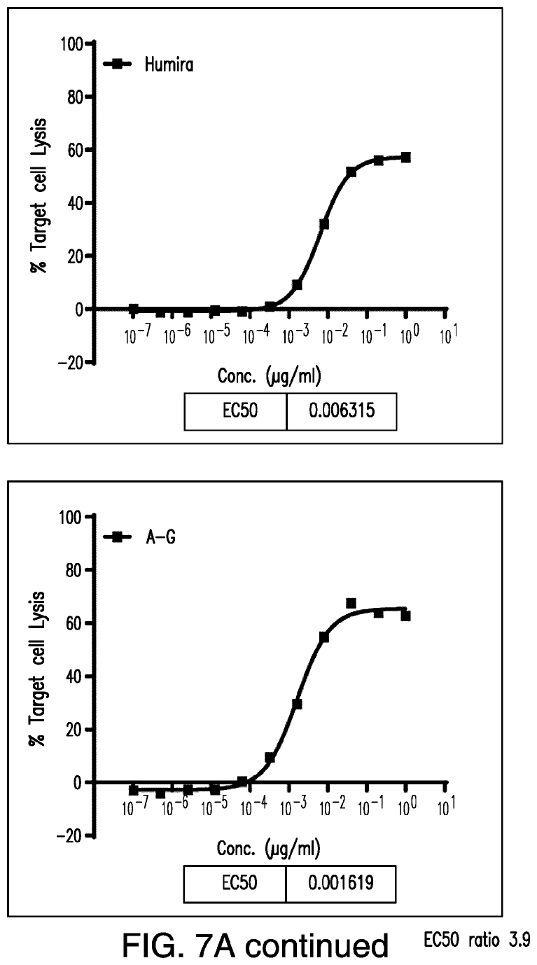

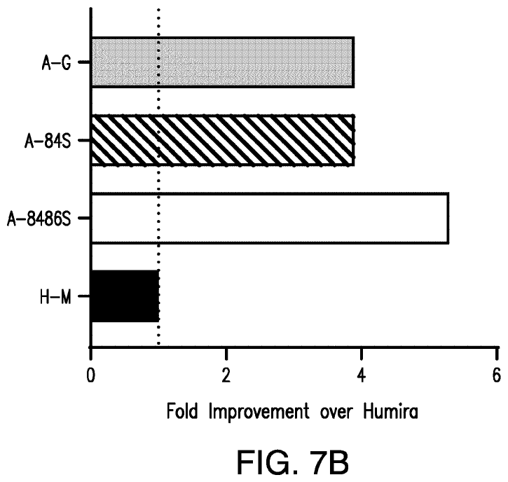

Purified human NK cells (E) from 1 donor were mixed with CHO cells expressing uncleavable membrane TNF (CHO-DG44/mTNF, T) at and E:T ratio of 5. Purity of NK cell was verified by flow cytometry staining for CD56 and CD3 and was above 90%. NK and CHO-DG44/mTNF cells were incubated for 6 hours at 37° C. with indicated dose response of antibodies. Target cell killing was measured by LDH release. The graphs of FIG. 7A show the dose response killing curve for indicated conditions. As the goal was to compare adalimumab glycovariant to adalimumab (HUMIRA), a HUMIRA condition was performed on each plate. EC50 values of killing were calculated in GraphPad Prism software using a log(agonist) vs. response, variable slope (four parameters) non-liner fitting. HUMIRA is adalimumab. Within FIG. 7A, the following can be seen: A-84S indicates an adalimumab variant with a sialylated N-glycan at position 84 of the variable heavy chain; A-8486S indicates an adalimumab variant with sialylated N-glycan at position 86 of the variable light chain and position 84 of the variable heavy chain; and A-G indicates an adalimumab in which the Fc N-glycan (position N-297) is a G0, this variant does not contain Fab N-glycans. The graph of FIG. 7B shows the ratio of EC50 values to HUMIRA EC50 for each adalimumab variants. This data show that A-84S and A-8486S Fab-sialylated adalimumab variants show a 4 to 5 fold enhanced ADCC compared to HUMIRA, similarly to an afucosylated variant in Fc only. Hence Fab sialylated glycans do not impair ADCC activity driven by their afucosylated Fc-glycan.

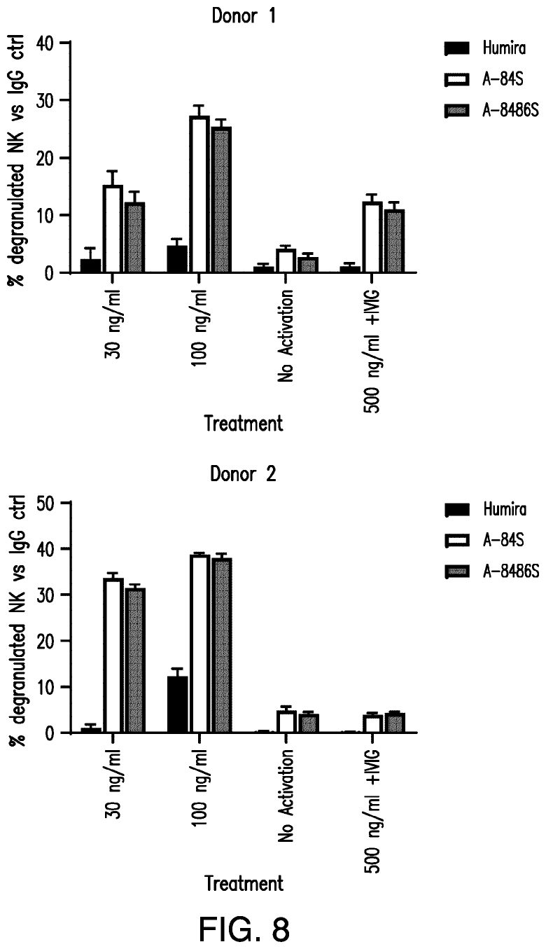

In a separate experiment, peripheral blood mononuclear cells (PBMCs) from 2 individual human donors were incubated for around 20h at 37° C., 5% CO2, in the presence of 150 U/ml IL-2, in RPMI-1640 supplemented with 10% FBS. Then 2×10⁶ PBMC were distributed to each assay point, and activated by adding TransAct CD3/CD28 beads (1:100) and LPS (1 μg/ml), in the presence of different glycovariants of adalimumab, or adalimumab (HUMIRA), as well as anti-CD107a antibody for the detection of degranulated NK cells. Samples were incubated at 37° C., 5% CO2 during 24h. Samples were stained with viability dye and anti-CD56, anti-CD4 and anti-CD8 to identify NK cells and samples were acquired by flow cytometry. Data was analyzed using FCS Express 6 (De Novo Software). Proportion of degranulated NK cells was determined through gating on live single cells, lymphocytes and eventually NK cells. Comparison between samples was done by Overton histogram subtraction. NK cell degranulation is a reliable readout for NK cell-mediated killing of target cells. The graphs of FIG. 8 show the average±SD of triplicates. Within FIG. 8, the following can be seen: A-84S indicates an adalimumab variant with a sialylated N-glycan at position 84 of the variable heavy chain; and A-84865 indicates an adalimumab variant with sialylated N-glycan at position 86 of the variable light chain and position 84 of the variable heavy chain; No Activation indicates that LPS and transact were not added and therefore minimal TNF production was ongoing; IVIG indicates a condition in which 1 mg/ml of pooled human Ig was added in the culture to compete with Fc receptors (this condition represents the closest to physiological condition). Antibodies were used at 30 ng/ml, 100 ng/ml and 500 ng/ml with IVIG. This data show that HUMIRA has very low ADCC, and undetectable ADCC activity in presence of competing human Ig. In contrast, both A-84S and A-8486S variants showed clear ADCC activity including in presence of competing Ig.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

### EXAMPLE IX

### Production and Quality Control of Adalimumab A-8486S Expressed in Cell Line St19866

A-8486S Fab-sialylated adalimumab was produced using CustomGlycan cell line St19866. The material was analyzed for product quality in comparison to HUMIRA with standard biochemical and high-resolution mass spectrometry methods. While the N-glycosylation profiles per design significantly differ between HUMIRA and A-8486S, biochemical properties of both antibodies should be highly comparable and the quality of A-8486S should match typical specification limits for monoclonal antibodies and the quality levels of commercial products.

A-8486S (batch P16-1658) was generated using cell line St19866 by application of standard protocols. The cell line contains glycoengineering elements such as those described in the International Patent Application Publications WO 2019/002512 and WO 2019/234021, which are incorporated herein by reference. Specifically, St19866 contains in one Pfr locus open reading frames for drMGAT1B, drMGAT2, rnMGAT1, hsB4GalT1, NeuC, CgNal, NeuB, hsST6, NeuA, rnMGAT2, hsMGAT1, and hsMGAT2, as well as in a second Pfr locus hsB4GALT1, rnMGAT2, gjMGAT1, and agMGAT1. In the ssu-Poll ribosomal DNA locus St19866 contains one construct comprising sfGNTI, drMGAT1B, rnMGAT2 , mmST6, CMAS, hsCST, another construct comprising rnMGAT1, hsMGAT1, gjMGAT1, and agMGAT1 as well as adalimumab K84N (heavy chain)/D86N (light chain). Additionally, the cell line is modified to prevent formation of the 0-linked GlcNAc by knock-out of three N-acetylglucosamine (GlcNAc)-transferases as described in WO 2021/140143, which is incorporated herein by reference. Table 3 provides the quality control parameters of A-8486S (batch P16-1658) compared to HUMIRA.

TABLE 3
Overview of A-8486S (batch P16-1658) quality assessment and targeted
specifications

| | | | A-8486S | |
|---|---|---|---|---|
| | | | St19866 | |
| Parameter | Assay | Specification/target | P16-1658 | HUMIRA |
| Purity/Integrity | SE-HPLC | Monomeric mAb ≥95% | 97.1% | 98.5% |
| | | HMW (aggregates) <5% | 0.6% | 0.9% |
| | | Shoulder (108 kDa): report results | 2.3% | 0.6% |
| | | LMW: report | 0.03% | 0% |
| | Non-red. PAGE | Main band ≥90% | 95% | 97% |
| | Red. PAGE | HC + LC ≥95% | 99% | 99% |
| Stability-3× F/T | SEC | <5% HMW | Monomeric: | Monomeric: |
| | | | 95.9%; | 98.0%; |
| | | | HMW: 0.7% | HMW: 0.6% |
| Stability-37° C.; 10 d | SEC | comparable to HUMIRA | Monomeric: | Monomeric: |
| | | | 95.5%; | 96.6%; |
| | | | HMW: 0.7% | HMW: 0.4% |
| N-glycan | HILIC-UPLC | Fab: A2G2S2 + A2G2S1 ≥70% | 76% | N/A |
| | | Fab: A2G2S2 ≥40% | 48% | N/A |
| | | Fab: Man3 ≤10% | 0.1% | N/A |
| | | Fc: A2 ≥40% | 52% | N/A |
| | | Fc: Man3 ≤10% | 2% | N/A |
| Endotoxin | LAL | ≤0.5 EU/mg | 0.180 | <0.05 |
| Human TNFα binding | ELISA | Rel. binding: 70-130% | 86% | 100% |
| | SPR | Rel. binding affinity: 70-130% | 84% | 100% |
| | IC formation by | Report results | Yes | Yes |
| | AF4-MALLS | | | |
| Macaque TNFα binding | ELISA | Rel. binding: 70-130% | 99% | 100% |
| | SPR | Rel. binding affinity: 70-130% | 77% | 100% |
| Human TNFα | L929 apoptosis | Rel. IC₅₀: 70-130% | 89% | 100% |
| neutralization | | | | |
| Complement | CDC on CHO— | Rel EC₅₀ killing: 70%-130% | 83% | 100% |
| dependent cytotoxicity | tmTNFα | | | |

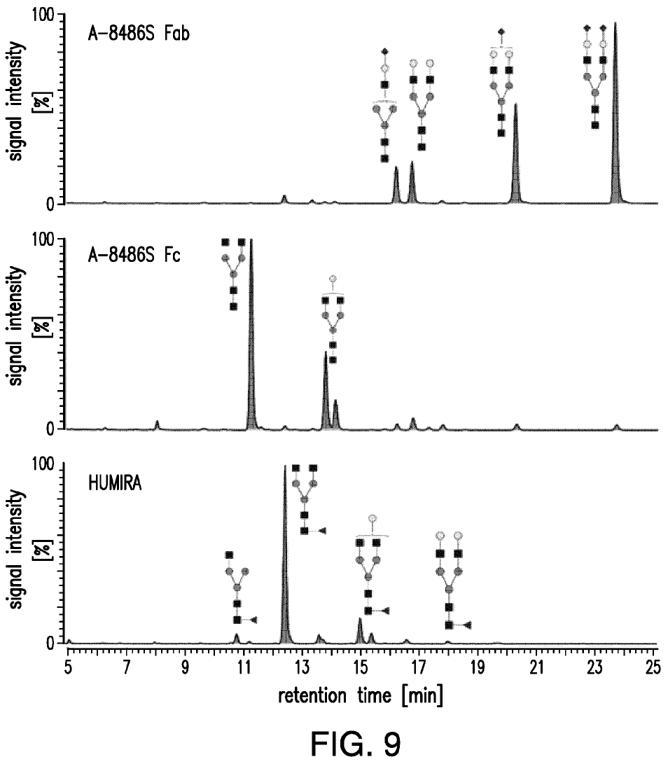

To quantitatively assess the N-glycosylation profiles of A-8486S (batch P16-1658) and HUMIRA, N-glycans were enzymatically released using PNGase F, fluorescently labelled with Procainamide and separated by HILIC-UPLC coupled to an electrospray mass spectrometer. In the case of A-8486S, IdeZ digestion and SDS-PAGE prior to glycan release and labelling were employed to gain individual profiles of the Fab N-glycans (K84N and D86N) and the Fc N-glycans. HUMIRA only contains the conserved Fc N-glycosylation site. As it can be seen in FIG. 9 and Table 4, Fab N-glycosylation of A-8486S (batch P16-1658) is dominated by sialylated N-glycans (86%) with A2G2S2 being the most abundant glycan structure (48%). The most abundant N-glycan present on the Fc N-glycosylation site of A-8486S (batch P16-1658) is A2 (52%). While N-glycans of A-8486S is by design fucose-free, the profile of the HUMIRA Fc glycosylation site is largely dominated by fucosylated N-glycans (total of 92%).

TABLE 4
Relative abundances of identified N-glycans on A-8486S (batch P16-1658) total, Fab, Fc and HUMIRA.
1: Total others refers to glycans not listed in table individually that have a relative abundance <0.5%.
Sialic acids in A-8486S are all α-2,6-linked.

| | Rel. abundance in % | | | | |
|---|---|---|---|---|---|
| N-glycan | A-8486S (P16- | A-8486S (P16- | A-8486S (P16- | N-glycan | Rel. abundance in % |
| structure | 1658) total | 1658) 201 Fab | 1658) Fc | structure | HUMIRA |
| M3 | 0.7 | 0.1 | 1.9 | FA1 | 3.5 |
| A1 | 0.4 | 0.3 | 0.4 | A2 | 0.7 |
| A2 | 17.6 | 0.1 | 51.6 | FA2 | 71.3 |
| A1G1 | 1.8 | 1.9 | 1.1 | M5 | 5.1 |
| M2A1G1S1 | 0.6 | 0.8 | 0.3 | FA2G1 | 15.8 |
| A2G1 | 12.0 | 0.8 | 32.4 | M6 | 2.1 |
| A1G1S1 | 6.7 | 9.0 | 1.9 | FA2G2 | 1.1 |
| A2G2 | 9.3 | 10.6 | 3.8 | Total others1 | 0.4 |
| A2G1S1 | 1.4 | 0.7 | 2.4 | | |
| A2G2S1 | 18.5 | 27.1 | 2.0 | | |
| A2G2S2 | 30.8 | 48.4 | 1.8 | | |
| Total others1 | 0.2 | 0.2 | 0.4 | | |

N-glycosylation occupancy on the three individual glycosylation sites on A-8486S (P16-1658), i.e. Fc N297 (HC), Fab N84 (HC) and Fab N86 (LC) was assessed by performing deglycosylation using PNGase F of a tryptic antibody digestion preparation in heavy water (H2O18). Briefly, this leads to the incorporation of O18 on the site of deglycosylation, where Asn is converted to Asp. Thus, it is possible to differentiate between unoccupied, deamidated (+1 Da) and deglycosylated (+3 Da) N-glycosylation sites. Samples were analyzed by LC-ESI MS and data was evaluated manually as well as using Byos (Proteinmetrics). Relative abundances were calculated by the ratio of the areas of the extracted ion chromatograms for the unmodified and deglycosylated peptides as obtained by LC-ESI MS. No deamidated peptides were observed for any of the listed N-glycosylation sites. As it can be seen in Table 5, the site occupancy is very high on all sites. The N84 site on the Fab heavy chain is the one with the lowest observed occupancy (94%).

TABLE 5
N-glycosylation site occupancy.

| | Fc N297 (HC) | Fab N84 (HC) | Fab N86 (LC) |
|---|---|---|---|
| A-8486S (P16-1658) | 99% | 94% | 98% |
| HUMIRA | 99% | N/A | N/A |

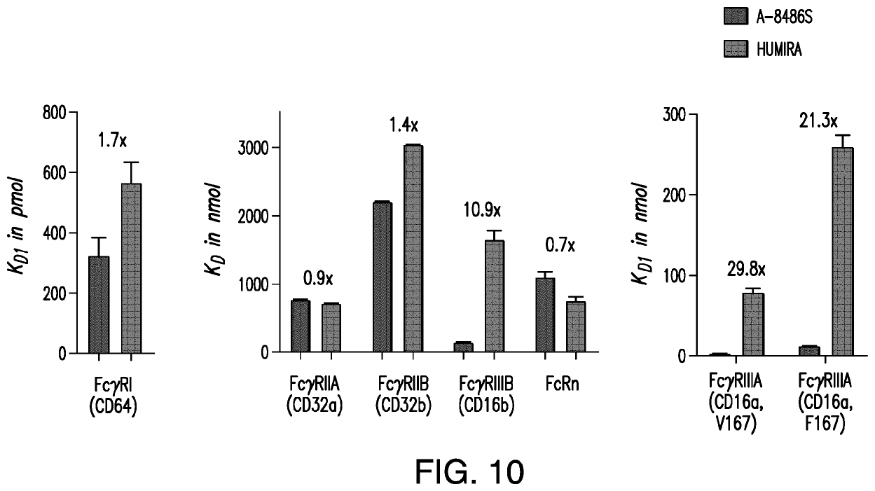

Binding to a panel of relevant Fc receptors was analyzed by SPR. K_{D} values and respective rel. binding affinities (calculated K_{D}(HUMIRA)/Ku(A-8486S)*100) as well as fold changes (calculated K_{D} (HUMIRA)/K_{D}(A-8486S) for FcγRIIA, FcγRIIB, FcRn were estimated using a steady state model, while for FcγRI, FcγRIIIA and FcγRIIIB a heterogenous ligand model was assumed. The latter resulted in two K_{D} values with the first one being the more meaningful one. Binding affinities of A-8486S (P 16-1658) to FcγRIII receptors are higher by 11-30 fold when comparing it to HUMIRA (see FIG. 10/Table 6).

TABLE 6
Binding affinity to a panel of Fc receptors.

| | | A-8486S (P16- | Humira | Rel. binding | |
|---|---|---|---|---|---|
| Fc Receptor | | 1658) K_{D}(M) | K_{D}(M) | affinity | Fold change |
| FcγRI | Mean K_{D1} | 3.26 E−10 | 5.66 E−10 | 174% | 1.7 |
| (CD64) | +/−SD | 0.56 E−10 | 0.67 E−10 | | |
| | Mean K_{D2} | 6.27 E−09 | 6.86 E−09 | 109% | 1.1 |
| | +/−SD | 0.28 E−09 | 0.69 E−09 | | |
| FcγRIIA | Mean | 7.85 E−07 | 7.23 E−07 | 92% | 0.9 |
| (CD32a, H167) | +/−SD | 0.06 E−07 | 0.09 E−07 | | |
| FcγRIIB | Mean | 2.20 E−06 | 3.04 E−06 | 138% | 1.4 |
| (CD32b) | +/−SD | 0.03 E−06 | 0.01 E−06 | | |
| FcγRIIIA | Mean K_{D1} | 2.69 E−09 | 8.01 E−08 | 2978% | 29.8 |
| (CD16a, V167) | +/−SD | 0.10 E−09 | 0.50 E−08 | | |
| | Mean K_{D2} | 2.74 E−08 | 7.66 E−08 | 280% | 2.8 |
| | +/−SD | 0.06 E−09 | 2.13 E−08 | | |
| FcγRIIIA | Mean K_{D1} | 1.22 E−08 | 2.60 E−07 | 2131% | 21.3 |
| (CD16a, F167) | +/−SD | 0.09 E−08 | 0.14 E−07 | | |
| | Mean K_{D2} | 1.23 E−07 | 1.11 E−06 | 902% | 9.0 |
| | +/−SD | 0.05 E−07 | 0.20 E−07 | | |
| FcγRIIIB | Mean | 1.52 E−07 | 1.65 E−06 | 1086% | 10.9 |
| (CD16b) | +/−SD | 0.03 E−07 | 0.14 E−06 | | |
| FcRn | Mean | 1.10 E−06 | 7.78 E−07 | 71% | 0.7 |
| (FCGRT&B2M) | +/−SD | 0.08 E−06 | 0.52 E−07 | | |

### EXAMPLE X

### Improved Fab Sialylation Cell Lines StCGP02824 and StCGP02826

Cell lines StCGP02824 and StCGP02826 were created to provide improved Fc N-glycan conversion and Fab sialylation. They contain glycoengineering elements such as those described in the International Patent Application Publications WO 2019/002512 and WO 2019/234021, which are incorporated herein by reference. Specifically, StCGP02824 contains in one Pfr locus open reading frames for drMGAT1B, drMGAT2, rnMGAT1, hsB4GalT1, NeuC, CgNal, NeuB, hsST6, NeuA, rnMGAT2, hsMGAT1, and hsMGAT2, in a second Pfr locus hsB4GALT1, rnMGAT2, gjMGAT1, and agMGAT1. In the ssu-PolI ribosomal DNA locus StCGP02824 contains sfGNTI, drMGAT1B, rnMGAT2 , mmST6, CMAS, and hsCST, as well as adalimumab K84N (heavy chain)/D86N (light chain). Additionally, the cell line is modified to prevent formation of the O-linked GlcNAc by knock-out of three N-acetylglucosamine (GlcNAc)-transferases as described in WO 2021/140143, which is incorporated herein by reference. StCGP02826 contains in one Pfr locus open reading frames for drMGAT1B, drMGAT2, rnMGAT1, hsB4GalT1, NeuC, CgNal, NeuB, hsST6, NeuA, rnMGAT2, hsMGAT1, and hsMGAT2, in a second Pfr locus hsB4GALT1, rnMGAT2, gjMGAT1, and agMGAT1. In the ssu-PolI ribosomal DNA locus StCGP02826 contains sfGNTI, drMGAT1B, rnMGAT2 , mmST6, CMAS, hsCST, and hsNGT, as well as adalimumab K84N (heavy chain)/D86N (light chain). Additionally, as for StCGP02826, the cell line is modified to prevent formation of the O-linked GlcNAc by knock-out of three N-acetylglucosamine (GlcNAc)-transferases.

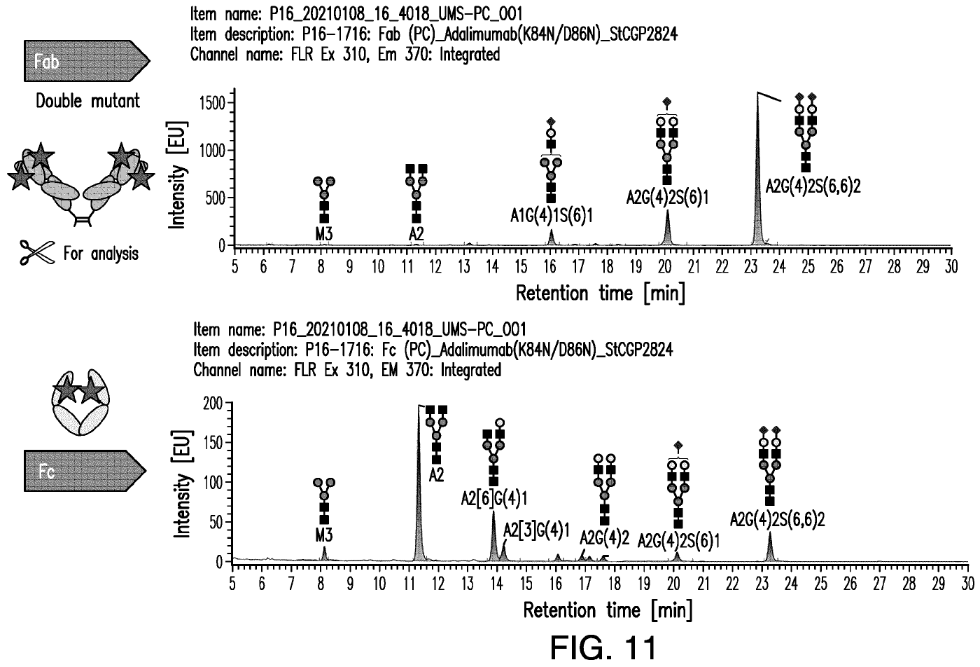

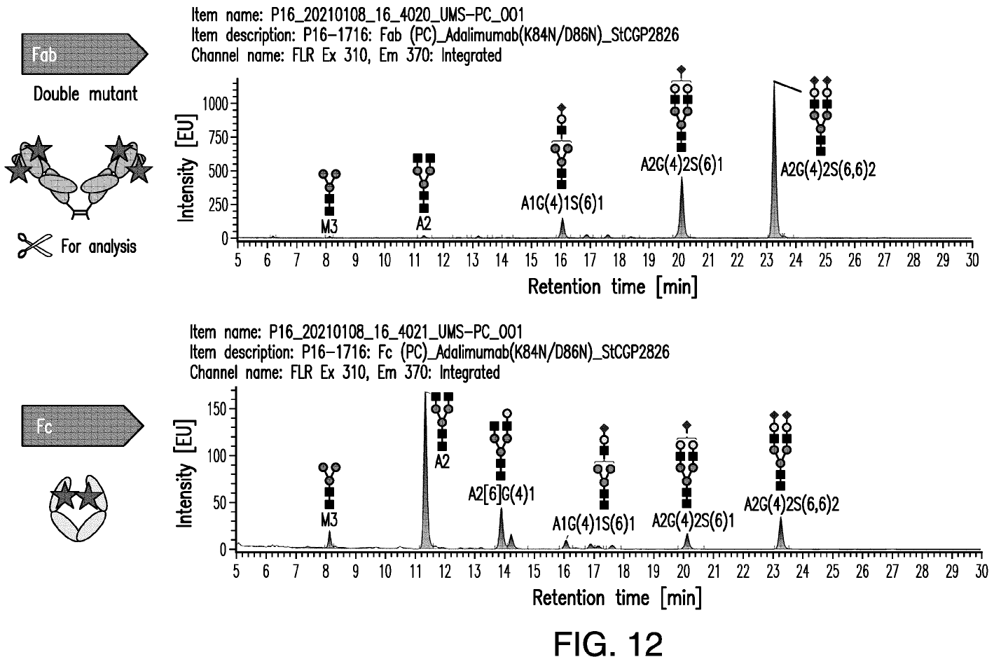

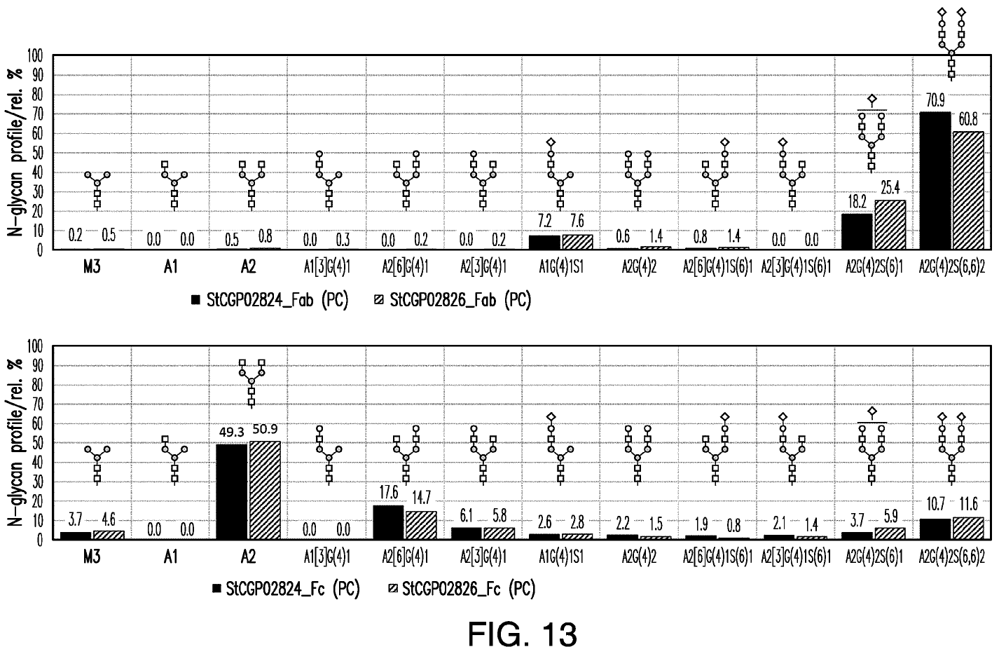

Adalimumab K84N-D86N was purified by Protein A from shake flask derived cell culture supernatant. The relative abundance of N-glycans on antibodies expressed in StCGP02824 and StCGP02826 was determined using the protocols described in Example 2. As shown in FIG. 11, N-glycans of the Fab glycosylation site of adalimumab K84N-D86N present very high abundance of alpha 2,6 biantennary sialylation and N-glycans of conserved N-glycosite 297 in the Fc part after expression in StCGP02824 present only minor amounts of the native M3 glycoform with most N-glycans being converted to A2. FIG. 12 exhibits a similar N-glycan profile for adalimumab K84N-D86N derived from StCGP02826. The top panel of FIG. 13 shows the relative quantification of glycoforms released from the Fab glycosites (K84N-D86N) of adalimumab expressed in StCGP02824 or StCGP02826, respectively. The bottom panel of FIG. 13 shows the corresponding conserved Fc glycosites, after separation by IdeZ cleavage. Nomenclature and glycan annotation is found in Table 2. With 60.8% and 70.9%, both cell lines demonstrate significantly higher abundance of alpha 2,6 biantennary sialylation on the Fab glycosites of adalimumab K84N-D86N and a strongly improved conversion of the M3 glycoform (3.7 and 4.6% remaining) on the Fc glycosite compared to the data shown in EXAMPLE IV.

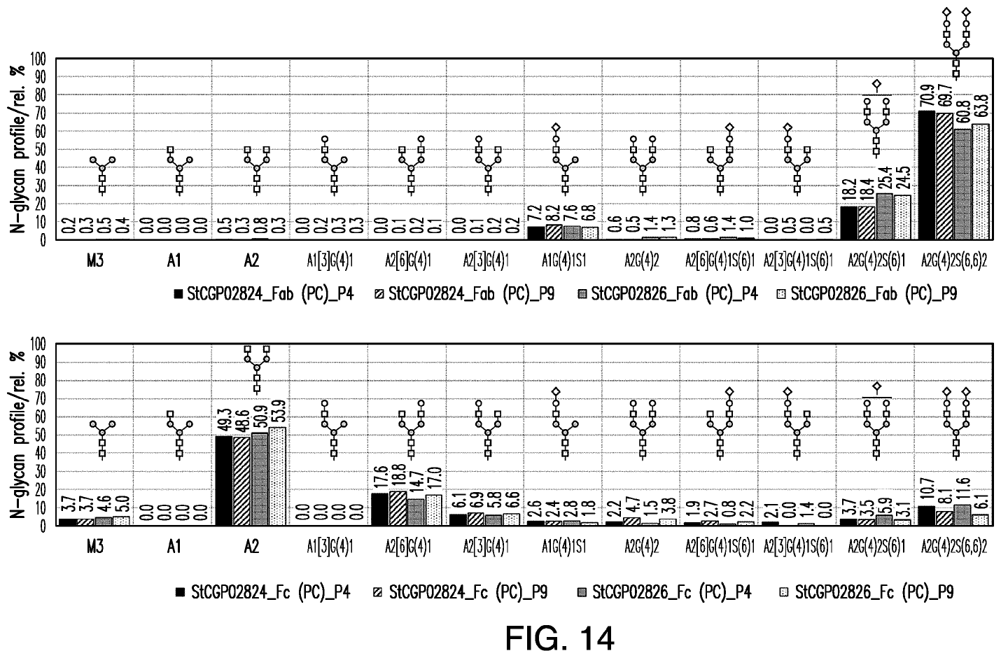

To assess stability of the glycoengineering strains, adalimumab was purified by Protein A from the two cell lines after 4 (P4) and 9 (P9) passages. The N-glycans released from the respective Fab and Fc glycosites exhibit almost identical profiles after prolonged maintenance of the cell line (FIG. 14).

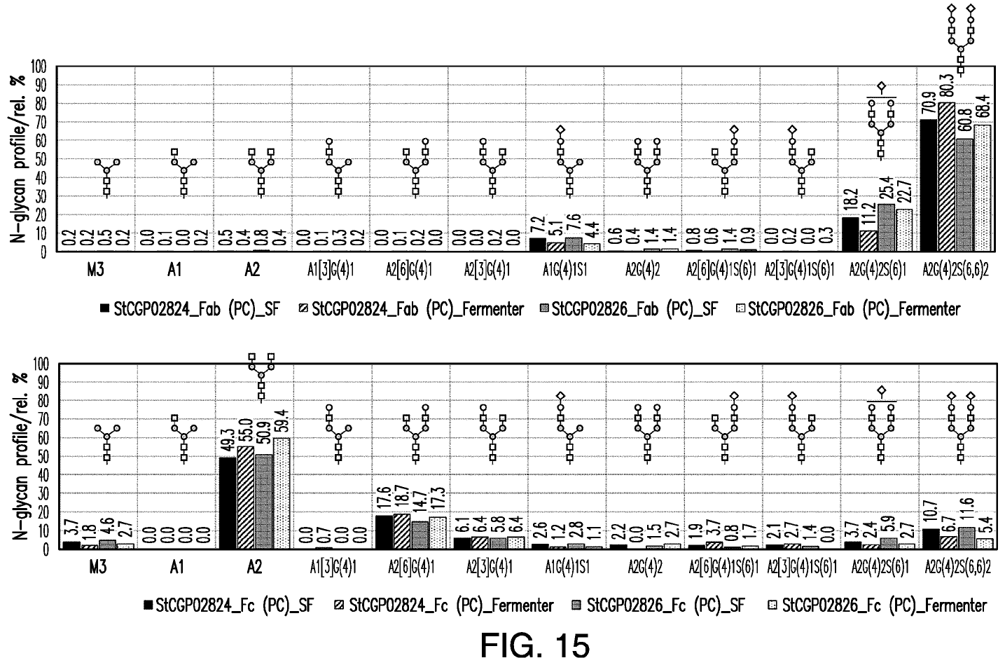

StCGP02824 and StCGP02826 were subjected to fed-batch fermentation in a DASbox mini bioreactor system using yeast extract based medium. Both strains exhibited stable growth and reached a maximum OD of 36 and 37 at the end of fermentation. Adalimumab (A-8486S) purified from cell culture supernatant was isolated by Protein A and a specific yield of 0.831 μg/OD and 1.05 μg/OD was determined. The N-glycan profile of the respective Fab and Fc glycosites are compared to those obtained upon growth of the same strains in shake flask (FIG. 15) and show even higher abundance of alpha 2,6 biantennary sialylation on the Fab glycosite as well as increased abundance of A2 on the Fc glycosite.

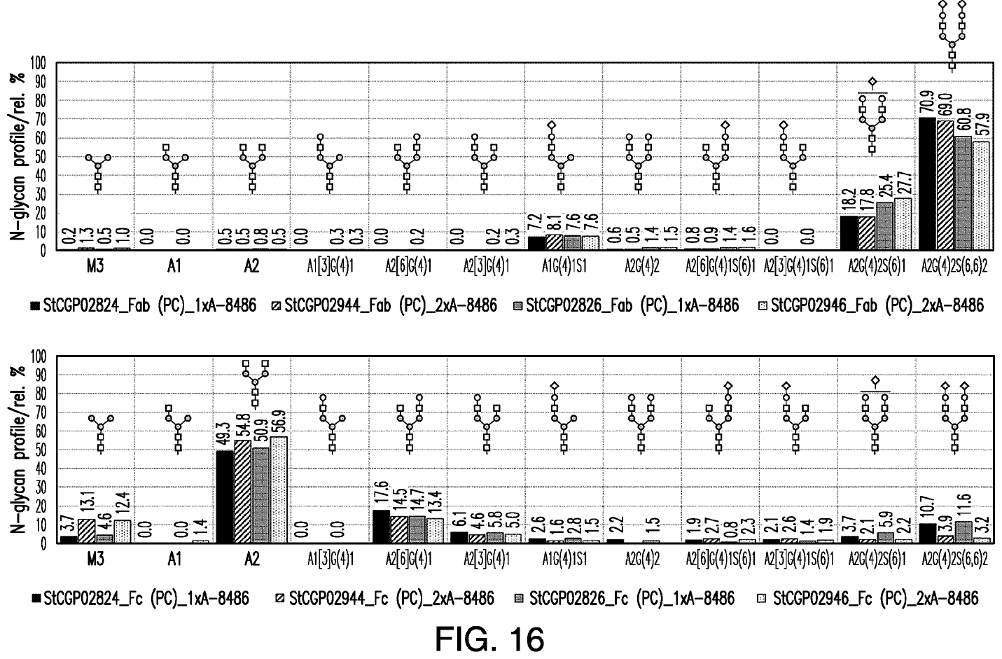

To assess the glycoengineering capacity of strains StCGP02824 and StCGP02826, additional genetic copies of adalimumab K84N-D86N were integrated into the ssu-Poll ribosomal DNA locus of these strains, creating strains StCGP02944 and StCGP02946, respectively. The adalimumab yield of these strains increased from 0.71 μg/OD (StCGP02824) to 2.53 μg/OD (StCGP02944) and from 0.93 μg/OD (StCGP02826) to 2.34 μg/OD (StCGP02946). Adalimumab was purified by Protein A from the new cell lines grown in shake flask and the N-glycans released from the respective Fab and Fc glycosites were compared with those obtained from the parental strains (FIG. 16). The N-glycan profiles remain very similar after yield increase with almost no change in abundance of alpha 2,6 sialylation on the Fab glycosites and only a small drop in conversion of the Fc N-glycan with 13.1% and 12.4% M3 remaining.

TABLE 7
N-glycans nomenclature and abbreviations

| | Oxford/Unifi | Short IgG | Symbol Nomenclature for Glycan |
|---|---|---|---|
| | nomenclature¹ | nomenclature² | structure³ |
| 1 | M3 | Man3 | 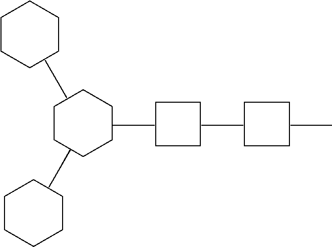 |
| 2 | A1 | G0-N | 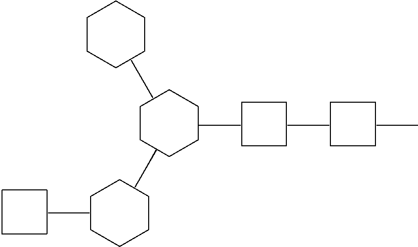 |
| 3 | A1G1 | G1-N | 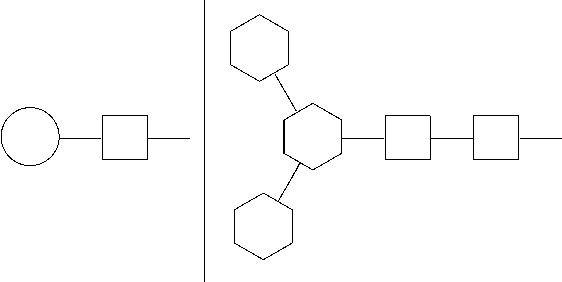 |
| 4 | A1[3]G(4)1 | G1-N | 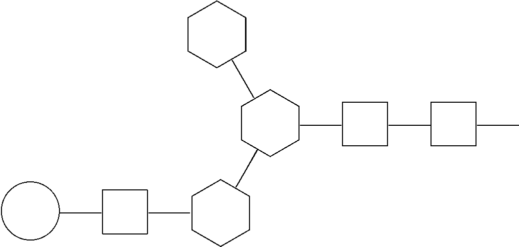 |
| 5 | A2 | G0 | 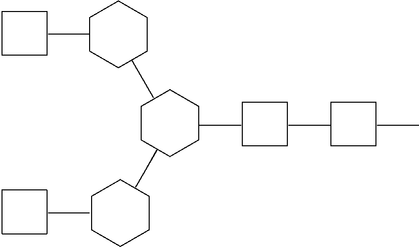 |
| 6 | A2[6]G(4)1 | G1(6′) | 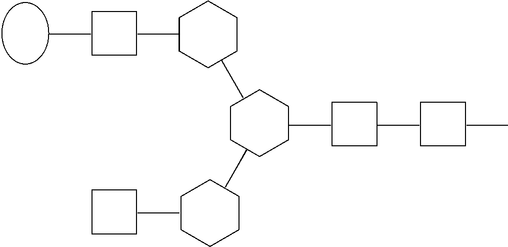 |
| 7 | A2[3]G(4)1 | G1(3′) | 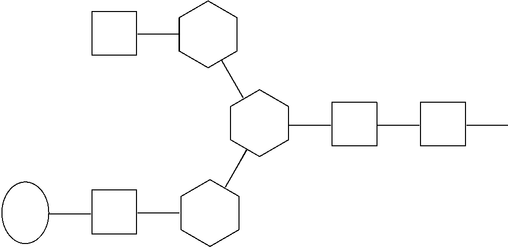 |
| 8 | A2G1 | G1 | 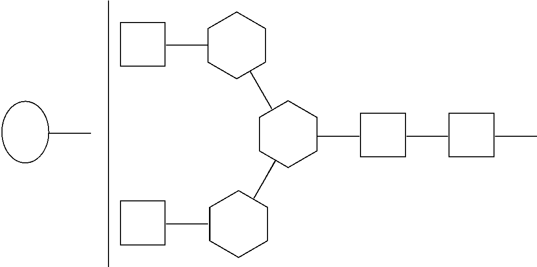 |
| 9 | A1G1S1 | G1S1-N | 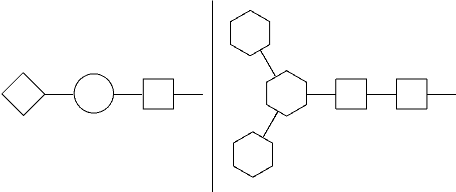 |
| 10 | A1[3]G(4)1S1 | G1S1-N | 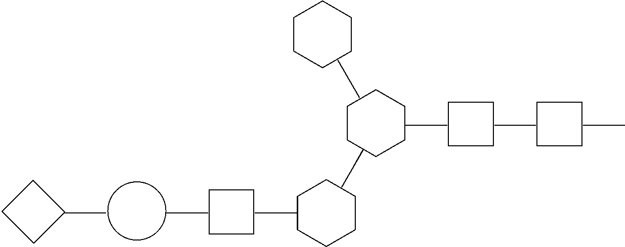 |
| 11 | A2G(4)2 | G2 | 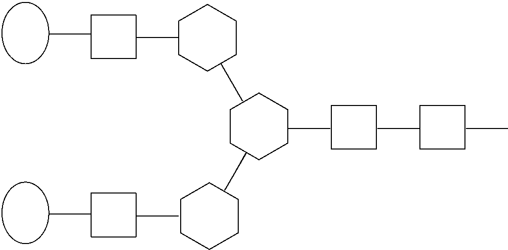 |
| 12 | A2G1S1 | G1S1 | 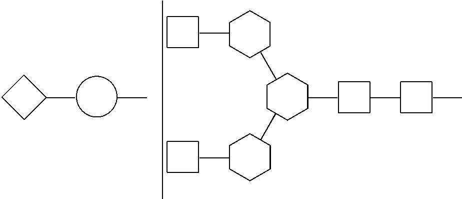 |
| 13 | A2[6]G(4)1S(6)1 | G1S1 | 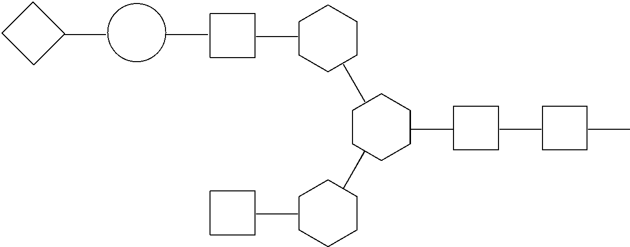 |
| 14 | A2[3]G(4)1S(6)1 | G1S1 | 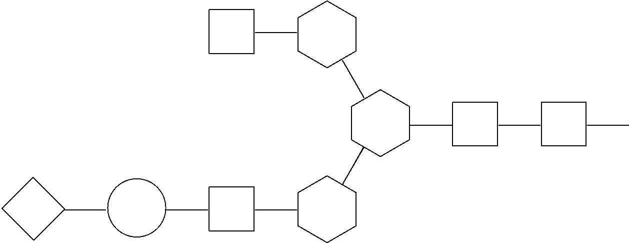 |
| 15 | A2G2S1 | G2S1 | 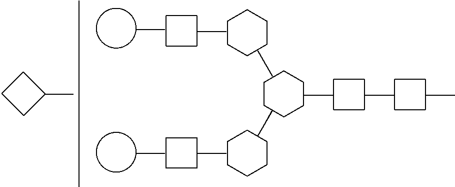 |
| 16 | A2G2S2 | G2S2 | 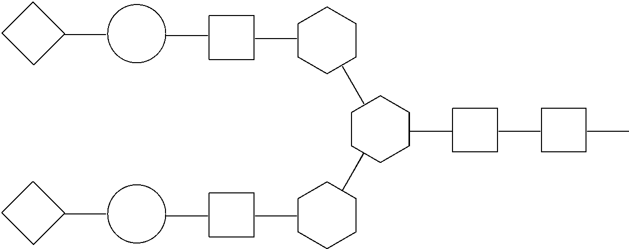 |
| 17 | A2G(4)2S(6,6)2 | G2S2 | 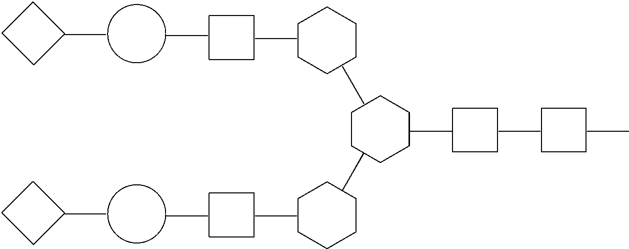 |

¹Mx: number (x) of residues within the oligomannose series;
Ax: number (x) of antennae;
F: core fucose;
Gx: number (x) of galactoses;
B: bisecting GlcNAc;
S: number (x) of sialic acids.
Note:
Linkage information is given in ( ) parentheses if applicable, e.g. F(6)A2 − α1-6 linked fucose. A2G1S1(6) − α2-6 linked sialic acid. Brackets [x] before G indicate which arm of the mannosyl core is galactosylated e.g. [3]G1 indicates that the galactose is on the antenna of the α1-3 mannose.
²This typically with IgG associated naming system indicates the presence of core fucose, the number of galactoses and the presence of biantennary glycans. It is limited in the number of structures and linkages it can describe but is often used for simplicity.
³Hexagon represents mannose (Man), white square is N-acetyl glucosamine (GlcNAc), white circle is galactose (Gal), white diamond is sialic acid, N-acetyl neuraminic acid (Neu5Ac).

### EXAMPLE XI

### Non Human Primate Study Demonstrate Safety of A-8486S and Trend for Reduced Immunogenicity

Next, A-8486S, produced in St19866, was tested in non human primate (NHP) cynomolgous monkey in a non-GLP pharmacology study. The quality attributes of A-8486S used in the NHP study are described in example IX. The objectives of this study were to characterize the pharmacokinetics, pharmacodynamics, safety and tolerability of A-8486S, when administered to the female Cynomolgus monkey on three occasions (Days 1, 8 and 15) via intravenous (bolus) injection at a dose level of 3 mg/kg. The effects observed were compared with the effect of the reference item, Humira (AbbVie), at the same dose level of 3 mg/kg. The dose of 3 mg/kg was chosen because it is equivalent to the Humira loading dose (160 mg) given to IBD patients. For this purpose, 12 female Cynomolgus monkeys were distributed into two experimental groups, of 6 animals each. Group A received the reference item (Humira), while Group B received the test item (A-8486S). The safety assessment relied on the evaluation of clinical pathology determinations conducted before starting the administrations and at different time points through the study, as well as on observed mortality, clinical signs, body temperature, blood pressure, body weight and food consumption of all the animals during the whole study. In addition, different samples were taken throughout the study in order to evaluate the pharmacokinetic profile of the test item as well as to do anti-drug antibody (ADA) analysis and cytokine analysis.

Treatment with A-8486S and Humira was well tolerated by all the animals. No mortality was observed over the study and only transient alterations in feces consistency were sporadically observed. No treatment related effects on body weight (Table 10), rectal temperature (Table 8), blood pressure (Table 9) and food consumption (Not shown) were observed in any experimental group.

Repeated administration of A-8486S and Humira, promoted an increase of absolute eosinophils count, absolute basophils count and reticulocyte count. The changes observed were very similar in A-8486S and Humira treated animals, as shown in Table 11. These changes correspond to described pharmacological effect of anti-TNF antibodies. Coagulation parameters, as measured by prothrombin time and activated partial thromboplastin time were not affected by A-8486S or Humira administration (Table 12).

TABLE 8
Summary of rectal temperature after each dosing.

| | | Week 1 (Day1) | | | Week 2 (Day 8) | | | Week 3 (Day 15) | | |
|---|---|---|---|---|---|---|---|---|---|---|
| | Week −1 | Predose | 0.5 h | 6 h | Predose | 0.5 h | 6 h | Predose | 0.5 h | 6 h |
| Humira | 37.85 ± | 38.68 ± | 38.55 ± | 38.18 ± | 38.52 ± | 38.13 ± | 37.80 ± | 38.38 ± | 37.77 ± | 37.88 ± |
| | 0.3 | 0.4 | 0.3 | 0.3 | 0.3 | 0.5 | 0.6 | 0.2 | 0.5 | 0.3α |
| A-8486S | 38.23 ± | 38.50 ± | 38.47 ± | 38.27 ± | 38.28 ± | 38.25 ± | 38.42 ± | 38.72 ± | 38.07 ± | 38.42 ± |
| | 0.3* | 0.6 | 0.5 | 0.6 | 0.4 | 0.3 | 0.4 | 0.8 | 0.7 | 0.9 |

Temperature are indicated as mean ° C. ± SD
* = Significantly different compared with Group A of the same week, p < 0.05
α = Significantly different compared with predose value from the same group on week 3 (p < 0.05)

TABLE 9
Summary of Summary of blood pressure.

| | | Day (hour post dose) | | | | | | | | |
|---|---|---|---|---|---|---|---|---|---|---|
| | | −4 | 1 (PrDs) | 1 (0.5 h) | 1 (6 h) | 8 (PrDs) | 8 (0.5 h) | 8 (6 h) | 15 (PrDs) | 15 (0.5 h) |
| Humira | Mean | 65.94 | 73.94 | 74.50 | 71.17 | 71.28 | 73.72 | 74.50 | 65.83 | 71.17 |
| | SD | 11.480 | 11.979 | 11.411 | 11.195 | 6.510 | 9.349 | 13.530 | 5.932 | 7.429 |
| A-8486S | Mean | 72.33 | 66.89 | 72.33 | 76.89 | 64.78 | 74.33 | 68.00 | 60.61 | 70.50 |
| | SD | 6.501 | 7.524 | 9.454 | 7.114 | 9.067 | 8.636 | 7.622 | 7.172 | 9.025 |
| Humira | Mean | 121.89 | 125.33 | 131.06 | 122.78 | 121.67 | 127.39 | 131.28 | 124.78 | 122.89 |
| | SD | 16.257 | 17.470 | 13.225 | 12.942 | 10.013 | 7.212 | 15.253 | 4.834 | 14.599 |
| A-8486S | Mean | 124.89 | 126.11 | 126.50 | 128.56 | 119.83 | 131.11 | 120.83 | 119.06 | 120.28 |
| | SD | 8.090 | 10.831 | 7.918 | 4.283 | 9.069 | 7.506 | 12.033 | 9.751 | 9.792 |

| | | Day (hour post dose) | | | | | | | | |
|---|---|---|---|---|---|---|---|---|---|---|
| | | 15 (6 h) | 22 | 29 | 36 | 43 | 50 | 57 | 64 | |
| Humira | Mean | 70.89 | 70.61 | 70.06 | 66.44 | 68.67 | 70.33 | 73.94 | 71.78 | Diastolic |
| | SD | 8.074 | 12.128 | 11.068 | 4.627 | 9.138 | 5.865 | 6.020 | 6.178 | P |
| A-8486S | Mean | 73.17 | 73.00 | 73.06 | 72.17 | 67.44 | 67.17 | 65.39 * | 70.67 | |
| | SD | 9.779 | 15.779 | 12.765 | 7.777 | 12.488 | 11.826 | 6.901 | 10.912 | |
| Humira | Mean | 122.67 | 131.78 | 130.94 | 124.61 | 123.56 | 126.39 | 124.22 | 129.44 | Systolic |
| | SD | 7.342 | 10.782 | 13.141 | 7.915 | 11.546 | 6.820 | 7.999 | 4.893 | P |
| A-8486S | Mean | 124.89 | 130.11 | 122.28 | 119.89 | 116.33 | 125.11 | 122.67 | 126.72 | |
| | SD | 10.500 | 12.781 | 9.974 | 10.633 | 10.646 | 7.702 | 11.890 | 8.242 | |

Data shows the diastolic and systolic blood pressure in mmHg
PrDs indicates Prior dosing
* = p < 0.05, T-test.

TABLE 10
Summary of body weight data.

| Days | | 1 | 4 | 8 | 11 | 15 | 18 | 22 | 25 | 29 | 32 |
|---|---|---|---|---|---|---|---|---|---|---|---|
| Humira | Mean | 3.38 | 3.26 | 3.31 | 3.27 | 3.33 | 3.35 | 3.37 | 3.39 | 3.47 | 3.43 |
| | SD | 0.277 | 0.272 | 0.307 | 0.266 | 0.238 | 0.204 | 0.247 | 0.246 | 0.272 | 0.271 |
| A- | Mean | 3.27 | 3.22 | 3.18 | 3.18 | 3.20 | 3.20 | 3.21 | 3.27 | 3.27 | 3.25 |
| 8486S | SD | 0.154 | 0.186 | 0.167 | 0.141 | 0.169 | 0.178 | 0.144 | 0.166 | 0.174 | 0.173 |

| Days | | 36 | 39 | 43 | 46 | 50 | 53 | 57 | 60 | 64 |
|---|---|---|---|---|---|---|---|---|---|---|
| Humira | Mean | 3.44 | 3.44 | 3.47 | 3.45 | 3.42 | 3.68 | 3.52 | 3.58 | 3.44 |
| | SD | 0.304 | 0.259 | 0.295 | 0.311 | 0.277 | 0.385 | 0.351 | 0.379 | 0.319 |
| A- | Mean | 3.24 | 3.25 | 3.26 | 3.23 | 3.25 | 3.38 | 3.34 | 3.35 | 3.32 |
| 8486S | SD | 0.163 | 0.158 | 0.146 | 0.175 | 0.186 | 0.264 | 0.211 | 0.220 | 0.215 |

Data show the mean body weight in Kg and SD.
No statistical differences between mean BW of 2 groups at any time point.

TABLE 11
Summary of changes in absolute eosinophil and basophil counts and in reticulocyte % throughout study period.

| | | | Day 1 | | | | Day 15 |
|---|---|---|---|---|---|---|---|
| | Parameter | Day −4 | (6 h postdose) | Day 2 | Day 3 | Day 8 | (Predose) |
| Humira | EOS | 0.25 ± 0.11 | 0.33 ± 0.14 | 0.84 ± 0.42 | 0.65 ± 0.36 | 1.10 ± 0.46** | 1.51 ± 0.39*** |
| | BAS | 0.10 ± 0.04 | 0.15 ± 0.07 | 0.19 ± 0.08 | 0.23 ± 0.11* | 0.23 ± 0.05* | 0.21 ± 0.03* |
| | RET | 1.57 ± 0.32 | 1.33 ± 0.39 | 1.67 ± 0.35 | 2.30 ± 1.16 | 2.17 ± 0.37 | 2.73 ± 0.64 |
| A-8486S | EOS | 0.17 ± 0.12 | 0.23 ± 0.13 | 0.61 ± 0.37* | 0.38 ± 0.19 | 0.82 ± 0.39** | 1.32 ± 0.66*** |
| | BAS | 0.07 ± 0.04 | 0.12 ± 0.07 | 0.19 ± 0.05* | 0.19 ± 0.13 | 0.25 ± 0.09** | 0.25 ± 0.08** |
| | RET | 1.33 ± 0.33 | 1.73 ± 0.39 | 2.33 ± 0.84 | 2.97 ± 0.69* | 2.93 ± 1.12 | 2.73 ± 0.88 |

| | | Day 15 | | | | |
|---|---|---|---|---|---|---|
| | Parameter | (6 h postdose) | Day 16 | Day 17 | Day 22 | Day 64 |
| Humira | EOS | 0.54 ± 0.32 α** | 0.33 ± 0.15 | 0.45 ± 0.18 | 0.82 ± 0.33* | 0.43 ± 0.23 |
| | BAS | 018 ± 0.07 | 0.16 ± 0.06 | 0.19 ± 0.05 | 0.23 ± 0.07* | 0.19 ± 0.10 |
| | RET | 3.00 ± 1.62 | 2.97 ± 1.88 | 3.00 ± 1.10 | 5.67 ± 1.96** | 1.30 ± 0.56 |
| A-8486S | EOS | 0.35 ± 0.17 α** | 0.35 ± 0.24 | 0.41 ± 0.26 | 0.67 ± 0.39* | 0.30 ± 0.16 |
| | BAS | 0.15 ± 0.04 | 0.14 ± 0.11 | 0.15 ± 0.08 | 0.21 ± 0.08* | 0.11 ± 0.05 |
| | RET | 3.10 ± 1.08* | 3.23 ± 0.64** | 2.83 ± 0.39* | 5.20 ± 0.95*** | 1.40 ± 0.78 |

EOS: Eosinophils; BAS: Basophils; RET: Reticulocytes. The table shows mean ± SD values.
* = Significantly different compared with basal levels (Day −4), p < 0.05
** = Significantly different compared with basal levels (Day −4), p < 0.01
*** = Significantly different compared with basal levels (Day −4), p < 0.001
α = Significantly different compared with predose value from the same group on Day 15 (*p < 0.05; **p < 0.01, ***p < 0.001)) by T-Test.

TABLE 12
Summary of coagulation parameters.

| | | Time Days (hours post dose) | | | | | |
|---|---|---|---|---|---|---|---|
| | | −4 | 1 (6 h) | 2 | 3 | 8 | 15 (prD) |
| Humira | PrTT | 10.32 ± 0.82 | 11.20 ± 0.77 | 11.05 ± 0.57 | 11.35 ± 0.59 | 10.73 ± 1.01 | 10.78 ± 0.55 |
| | APTT | 37.58 ± 3.15 | 29.03 ± 4.05 | 30.37 ± 3.54 | 26.07 ± 2.78 | 24.68 ± 2.36 * | 39.95 ± 8.12 |
| A-8486S | PrTT | 12.88 ± 1.09 | 11.18 ± 0.78 | 10.63 ± 0.96 * | 11.47 ± 1.08 | 10.83 ± 0.62 * | 11.13 ± 1.08 |
| | APTT | 29.90 ± 7.79 | 28.93 ± 5.62 | 23.57 ± 4.94 | 29.28 ± 3.98 | 21.63 ± 1.70 | 36.64 ± 7.59 |

| | | Time Days (hours post dose) | | | | |
|---|---|---|---|---|---|---|
| | | 15 (6 h) | 16 | 17 | 22 | 64 |
| Humira | PrTT | 11.63 ± 0.52 ** & α* | 10.75 ± 0.75 | 11.22 ± 0.74 | 10.63 ± 0.48 | 10.72 ± 0.61 |
| | APTT | 25.67 ± 3.26 α** | 18.47 ± 2.66 **** | 27.97 ± 9.15 | 27.77 ± 4.38 | 26.08 ± 4.07 |
| A-8486S | PrTT | 11.72 ± 0.88 | 10.87 ± 0.97 | 11.38 ± 0.98 | 10.25 ± 0.98 * | 11.15 ± 0.82 |
| | APTT | 25.98 ± 4.53 α* | 18.52 ± 3.40 * | 23.12 ± 3.36 | 23.35 ± 5.49 | 26.58 ± 3.01 |

The table shows mean ± SD values.
PrTT: Prothrombin time (seconds); APTT: activated partial thromboplastin time (seconds); PrD: Predose.
* = Significantly different compared with basal levels (Day −4), p < 0.05
** = Significantly different compared with basal levels (Day −4), p < 0.01
*** = Significantly different compared with basal levels (Day −4), p < 0.001
**** = Significantly different compared with basal levels (Day −4), p < 0.0001
α = Significantly different compared with predose value from the same group on Day 15 (* p < 0.05; ** p < 0.01, *** p < 0.001))

Blood biochemistry parameters were not affected by the repeated administration of Humira or A-8486S. The following blood biochemistry parameters were measured: Albumin, Total proteins, Globulins, Cholesterol, Triglycerides, Glucose, Urea, Total bilirubin, Creatinine, Alkaline phosphatase, Alanine aminotransferase, Aspartate aminotransferase, Gamma glutamyltransferase, Electrolytes (Ca2+, Cl−, PO43−, K+, Na+).

The following cytokines in peripheral blood serum were analysed by ELISA using commercial kit following manufacturer's instruction: IL-2 (Mybiosource reference MB S761878), IL-6 (Abcam reference Ab242233), TNF-α (Abcam reference Ab252354), IFN-γ (Abcam reference Ab270895), IL-8 (Abcam reference Ab242232), IL-10 (Mybiosource reference MBS2501888). In addition, a positive control (plasma harvested from activated blood) was produced to be included in each analysis in order to ensure that the kits worked properly. The positive control was produced as follows. Whole blood was collected from one cynomolgous monkey female belonging to the service provider colony (animal not participating to the study). Approximately 6 mL of blood was collected into sodium heparin tubes to prevent coagulation. This whole blood was incubated with a mitogenic solution (LPS at 1 μg/ml final and phytohemagglutinin at 100 μg/ml final) for 24 hours in a 24 well plate at 37° C. and 5% CO2 on a plate shaker at 50-100 rpm. After the incubation time, the content of each well was poured into a falcon tube of 15 mL and centrifuged at 5° C. for 10 min at 2000g to pellet the cells and the plasma was harvested, aliquoted and stored at −80° C. until analysed. The cytokines were analysed at the following timepoints: Week −1 (predose baseline), Day 1 6 hours after 1^{st} dose, Day 8 (predose=7 days after 1^{st} dose), Day 15 (predose), Day 15 (6 hours post dose). None of the analysed cytokines showed significant elevation compared to predose levels at any timepoints showing that A-8486S did not induce measurable cytokine release. The Tables 13, 14 and 15 shows the values for IL-6, IL-8 and TNFα respectively.

TABLE 13
Individual IL-6 levels.

| | Animal | Week −1 | Day 1 | Day 8 | Day 15 | Day 15 |
|---|---|---|---|---|---|---|
| Group | ID | (basal) | (6 h) | (PrD) | (PrD) | (6 h) |
| Humira | 1 | <LLOD | <LSTD | <LSTD | <LLOQ | <LLOD |
| | 2 | <LLOQ | <LLOQ | 51.6 | <LLOQ | 49.6 |
| | 3 | <LSTD | 49.6 | <LSTD | <LSTD | <LSTD |
| | 4 | <LSTD | <LLOD | <LSTD | <LSTD | 48.4 |
| | 5 | <LLOD | <LSTD | <LLOQ | <LSTD | <LSTD |
| | 6 | <LLOD | <LLOQ | <LLOD | <LLOQ | <LLOQ |
| A-8486S | 8 | <LLOD | <LLOQ | 51.6 | <LLOQ | <LLOD |
| | 9 | <LLOQ | <LLOQ | <LLOQ | <LLOD | <LLOD |
| | 10 | <LSTD | <LLOQ | <LLOD | <LLOD | <LLOD |
| | 11 | <LLOD | <LLOD | <LLOQ | <LLOQ | <LLOD |
| | 12 | <LLOD | <LLOD | <LLOQ | <LSTD | <LSTD |

Table shows the individual IL-6 concentration in pg/ml.
LLOD = Lower limit of detection;
LLOQ = Lower limit of quantification;
LSTD = Lowest standard, 12.5 pg/mL

TABLE 14
Summary of IL-8 levels.

| Time (hour | Week −1 | | | | |
|---|---|---|---|---|---|
| post dose) | (basal) | Day 1 (6 h) | Day 8 (PrD) | Day 15 (PrD) | Day 15 (6 h) |
| Humira | 1297.0 ± 635.2 | 1521.7 ± 723.7 | 1871.8 ± 839.6 | 2503.6 ± 1322.1 | 2460.9 ± 1531.2 |
| A-8486S | 2256.6 ± 825.6* | 2259.5 ± 1008.0 | 2509.1 ± 1129.9 | 2580.3 ± 1347.8 | 3304.9 ± 926.2 |

Table shows mean ± SD values in pg/ml.
PrD: Pre-dose.
*= p < 0.05 by un-paired t-test vs Humira group

TABLE 15
Individual TNFα values.

| | Animal | Week −1 | Day 1 | Day 8 | Day 15 | Day 15 |
|---|---|---|---|---|---|---|
| Group | ID | (basal) | (6 h) | (PrD) | (PrD) | (6 h) |
| Humira | 1 | <LLOD | <LLOD | <LLOD | <LLOD | <LLOD |
| | 2 | <LLOD | <LLOD | <LLOD | <LLOD | <LLOD |
| | 3 | 1898.6 | 1552.9 | 1236.0 | 1189.8 | 1153.8 |
| | 4 | <LLOQ | <LLOD | <LLOD | <LLOQ | <LLOQ |
| | 5 | <LLOQ | <LLOQ | <LLOD | <LSTD | <LSTD |
| | 6 | <LLOD | <LLOD | <LSTD | <LLOQ | <LLOD |
| A-8486S | 8 | <LLOD | <LLOD | <LLOD | <LSTD | <LSTD |
| | 9 | 401.7 | 330.3 | 352.4 | 319.2 | 357.9 |
| | 10 | <LSTD | <LSTD | <LSTD | <LLOD | <LLOD |
| | 11 | <LLOD | <LLOD | <LLOD | 363.4 | 291.4 |
| | 12 | <LLOD | <LLOD | <LLOQ | <LLOD | <LSTD |

Table shows the individual IL-6 concentration in pg/ml.
LLOD = Lower limit of detection;
LLOQ = Lower limit of quantification;
LSTD = Lowest standard, 15.63 pg/mL

Pharmacokinetic analysis of A-8486S and Humira levels in peripheral blood was performed at different timepoints. Blood samples collected in clot activator tubes were left to clot at ambient temperature for at least 5 minutes. Tubes were then centrifuged at 2000 g during 10 min at 4° C. After centrifugation tubes were stored in an ice bath until serum separation. Serum samples were aliquoted and stored frozen at −80° C. until analysis. The bioanalysis of PK samples used an electrochemiluminescence-based (ECL, Mesoscale Discovery (MSD)) sandwich assay. Briefly, MSD Streptavidin-coated electrode plates were blocked with 3% BSA (blocking buffer) for at least 1.5 hours. Biotinylated anti-Adalimumab antibody (HCA202, Biorad) capture solution at 1 μg/ml was added to each well. It was verified during method development that HCA202 captures equally Humira and A-8486S. Calibrator curve set, quality control samples (QCs), study samples, and blank samples in 1% matrix were loaded to respective plate wells. SULFO-tagged anti-Adalimumab antibody (HCA204, Biorad) detection solution (2 μg/ml) was added to each well. MSD read buffer was added to each well and ECL signal was acquired using and MSD Sector Imager. Importantly, separate calibration curves were used for quantification of Humira and A-8486S (i.e. a Humira calibration curve was used for quantification of Humira in samples and a A-8486S calibration curve was used for quantification of A-8486S in samples). Toxicokinetic parameters were calculated using the validated application Phoenix WinNonlin® version 6.2.11. Non-compartmental analysis using model Plasma (200-202)—IV Bolus was applied. After 1st and 3rd dose, the PK parameters of A-8486S and Humira were highly comparable, as shown in Table 9. These data indicate that Fab sialylation of A-8486S did not adversely alter its PK profile.

TABLE 16
Summary of pharmacokinetic parameters of A-8486S and
Humira after 1st and 3rd dose.

| | PK Parameter | Humira | A-8486S |
|---|---|---|---|
| After 1^{st} Dose | C₀ (ng/ml) | 111’996 ± 6’283 | 91’825 ± 9’966 |
| | AUC₀₋ₗₐₛₜ | 9’003’934 ± 1’057’172 | 6’507’546 ± 614’220 |
| | (ng · h/ml) | | |
| | t½ (h) | [161.2 ± 89.0] | [149.1 ± 42.8] |
| | Cl (ml/h/kg) | [0.1946 ± 0.0417] | [0.2662 ± 0.0546] |
| After 3^{rd} Dose | C₀ (ng/ml) | 122’447 ± 19’560 | 104’852 ± 24’637 |
| | AUC₀₋ₗₐₛₜ | 5’665’259 ± 4’362’114 | 4’115’619 ± 3’026’295 |
| | (ng · h/ml) | | |
| | t½ (h) | [31.58] ± [18.55] | [30.72] ± [24.49] |
| | Cl (ml/h/kg) | [0.8189] ± [0.5721] | [1.275] ± [1.279] |

The table shows the average ± SD. The values noted as [value] indicate that accurate determination was not possible.
C₀: Extrapolated analyte plasma concentration at t = 0.
AUC₀₋ₗₐₛₜ: Area under the analyte vs time concentration curve from time of administration up to the time of the last quantifiable concentration, calculated by linear up/log down summation.
t½: The apparent terminal elimination half-life.
Cl: Systemic clearance, determined by Dose/AUC0-∞.

Anti-drug antibody (ADA) levels, against Humira and A-8486S was measured using an electrochemiluminescence based bridging assay (ECL, Mesoscale Discovery). Briefly, MSD electrode plates were coated with Humira or A-8486S overnight at 4° C. at 0.5 μg/ml in 1×PBS. Plates were blocked using 3% BSA (Blocking Buffer) for at least 1.5 hours. Calibrator samples, quality control samples (QCs), study samples, and blank samples were loaded in 1% Matrix to respective plate wells. SULFO-Tag conjugated Humira or A-8486S (Detection Solution at 1 μg/ml) were added to all wells. MSD Read Buffer was added to all wells. ECL signal was acquired using an MSD Sector Imager. ECL signal within the calibration curve were directly used as a semi quantitative representation of the ADA response against Humira and A-8486S. All determinations were based on duplicate analysis of each sample (i.e., 2 wells). The coefficient of variation percentage (% CV) was calculated between the signals of the two wells. The % CV needed to be <20% between duplicate wells per sample.

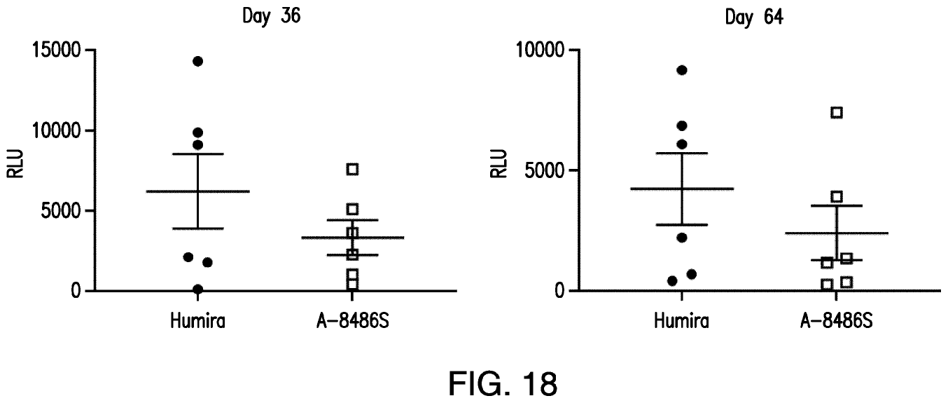

FIG. 18 shows the ADA data at day 36 and day 64 (termination) of the study. ADA levels were reduced by 50% in A-8486S group as compared to Humira group at day 36. The difference was not statistically significant (as determined by determined by an unpaired t test with Welch's correction) due to the low number of data points and the high variability of the ADA response. The trend for reduced ADA formation against A-8486S was confirmed at Day 64 but also not statistically significant. Within FIG. 18 the following can be seen: The left graph show the ADA levels at day 36 and right graph shows the ADA levels at study termination (day 64). RLU represent the relative luminescence unit (ECL signal). Each point represents the ADA level in one animal (black circles Humira and open squares A-8486S). The mean±SEM of each group are represented by the horizontal and vertical lines. These data support the hypothesis that Fab sialylation of A-8486S leads to a lower ADA response in monkey.

Overall the safety data, in combination with the PK data and immunogenicity data showed that A-8486S was well tolerated in cynomolgus monkey when injected at 3 weekly doses of 3 mg/kg, while showing a similar exposure than Humira injected at same dose. These data support the assumption that A-8486S with its afucosylated Fc-glycan leading to enhanced ADCC potency and enhanced M2 macrophage formation, and high sialylation of its Fab glycans, leading to reduced immunogenicity as compared to Humira, will display a similar safety profile than Humira and offer a higher efficacy in human patients.

SEQUENCE LISTING

| SEQ | | Other | |
|---|---|---|---|
| ID NO. | Description | name | Sequence |
| 1 | Human UDP- | hsNGT | MYPYDVPDYAYPYDVPDYAYPYDVPDYAGGSGGFANLKYVSLGILVFQ |
| | GlcNAc | | TTSLVLTMRYSRTLKEEGPRYLSSTAVVVAELLKIMACILLVYKDSKCSLR |
| | Transporter | | ALNRVLHDEILNKPMETLKLAIPSGIYTLQNNLLYVALSNLDAATYQVTY |
| | (Uniprot | | QLKILTTALFSVSMLSKKLGVYQWLSLVILMTGVAFVQWPSDSQLDSK |
| | Q9Y2D2), N- | | ELSAGSQFVGLMAVLTACFSSGFAGVYFEKILKETKQSVWIRNIQLGFF |
| | term 3xHA tag | | GSIFGLMGVYIYDGELVSKNGFFQGYNRLTWIVVVLQALGGLVIAAVIK |
| | | | YADNILKGFATSLSIILSTLISYFWLQDFVPTSVFFLGAILVITATFLYGYDP |
| | | | KPAGNPTKA |
| 2 | Beta- | d88hST6 | MIASSVRHAVILLLVAVAMMVNNVIAPEASFQVWNKDSSSKNLIPRLQ |
| | galactoside | | KIWKNYLSMNKYKVSYKGPGPGIKFSAEALRCHLRDHVNVSMVEVTD |
| | alpha-2, 6- | | FPFNTSEWEGYLPKESIRTKAGPWGRCAVVSSAGSLKSSQLGREIDDH |
| | sialyltransferase | | DAVLRFNGAPTANFQQDVGTKTTIRLMNSQLVTTEKRFLKDSLYNEGIL |
| | 1 (Uniprot | | IVWDPSVYHSDIPKWYQNPDYNFFNNYKTYRKLHPNQPFYILKPQMP |
| | P15907), aa89- | | WELWDILQEISPEEIQPNPPSSGMLGIIIMMTLCDQVDIYEFLPSKRKT |
| | 406, N- | | DVCYYYQKFFDSACTMGAYHPLLYEKNLVKHLNQGTDEDIYLLGKATL |
| | terminal | | PGFRTIHC |
| | synthetic | | |
| | signal peptide | | |
| 3 | CMP-Neu5Ac | mmCST- | MAPARENVSLFFKLYCLTVMTLVAAAYTVALRYTRTTAEELYF |
| | transporter | GFP | STTAVCITEVIKLLISVGLLAKETGSLGRFKASLSENVLGSPK |
| | (Q61420.2), | | ELAKLSVPSLVYAVQNNMAFLALSNLDAAVYQVTYQLKIPCTA |
| | GFP fusion | | LCTVLMLNRTLSKLQWISVFMLCGGVTLVQWKPAQATKVVVAQ |
| | | | NPLLGFGAIAIAVLCSGFAGVYFEKVLKSSDTSLWVRNIQMYL |
| | | | SGIVVTLAGTYLSDGAEIQEKGFFYGYTYYVWFVIFLASVGGL |
| | | | YTSVVVKYTDNIMKGFSAAAAIVLSTIASVLLFGLQITLSFAL |
| | | | GALLVCVSIYLYGLPRQDTTSIQQEATSKERIIGVGGSGGMSK |
| | | | GEELFTGVVPILVELDGDVNGHKFSVRGEGEGDATNGKLTLKF |
| | | | ICTTGKLPVPWPTLVTTLTYGVQCFSRYPDHMKRHDFFKSAMP |
| | | | EGYVQERTISFKDDGTYKTRAEVKFEGDTLVNRIELKGIDFKE |
| | | | DGNILGHKLEYNFNSHNVYITADKQKNGIKANFKIRHNVEDGS |
| | | | VQLADHYQQNTPIGDGPVLLPDNHYLSTQSVLSKDPNEKRDHM |
| | | | VLLEFVTAAGITHGMDELYK |

1. A monoclonal antibody comprising a higher amount of sialic acid in a Fab region of the monoclonal antibody as compared to an antibody found in human serum or a control monoclonal antibody produced by a Chinese hamster ovary (CHO) cell line.

2. A monoclonal antibody comprising a higher amount of sialic acid in a Fab region and/or a higher amount of sialic acid in an Fc region of the monoclonal antibody as compared to an antibody found in human serum or a control monoclonal antibody produced by a Chinese hamster ovary (CHO) cell line.

3. A monoclonal antibody comprising a higher amount of sialic acid in a Fab region and/or a higher amount of afucosylated glycan in an Fc region of the monoclonal antibody as compared to an antibody found in human serum or a control monoclonal antibody produced by a Chinese hamster ovary (CHO) cell line.

4. A monoclonal antibody comprising a higher amount of sialic acid in a Fab region and/or higher amount of G0 glycan in an Fc region of the monoclonal antibody as compared to an antibody found in human serum or a control monoclonal antibody produced by a Chinese hamster ovary (CHO) cell line.

5. The monoclonal antibody of claim 1 comprising sialylated glycans at one or more point mutations in the variable domain of the heavy chain and/or light chain of the monoclonal antibody.

6. The monoclonal antibody of claim 1 comprising sialylated glycans at one or more inserted or mutated amino acids leading to:
a. an N-glycosylation site in the framework region of the variable domain of the heavy chain of the monoclonal antibody, wherein the monoclonal antibody retains its ability to bind its antigen; or
b. an N-glycosylation site in the framework region of the variable domain of the light chain of the monoclonal antibody, wherein the monoclonal antibody retains its ability to bind its antigen; or
c. an N-glycosylation site in the framework region of the variable domain of the heavy chain and sialylated glycans at one or more inserted or mutated amino acids leading to an N-glycosylation site in the framework region on the variable domain of the light chain, wherein the monoclonal antibody retains its ability to bind its antigen.

7. (canceled)

8. (canceled)

9. The monoclonal antibody of claim 1 comprising afucosylated glycan structures (i) at the Fc region of the monoclonal antibody or (ii) at the conserved Fc glycosite N297 (according to IMGT database and Eu numbering).

10. (canceled)

11. The monoclonal antibody of claim 1, wherein the monoclonal antibody is an anti-TNFα antibody.

12. The monoclonal antibody of claim 11, wherein the anti-TNFα antibody is a variant of adalimumab.

13. The monoclonal antibody of claim 12 comprising:
a. sialylated glycans at N84 (according to IMGT database numbering) on the variable domain of the heavy chain of the monoclonal antibody; or
b. sialylated glycans at N86 (according to IMGT database numbering) on the variable domain of the light chain of the monoclonal antibody; or
c. sialylated glycans at N84 (according to IMGT database numbering) on the variable domain of the heavy chain and sialylated glycans at N86 on the variable domain of the light chain of the monoclonal antibody.

14. (canceled)

15. (canceled)

16. The monoclonal antibody of claim 1, comprising one or more of the following structures: 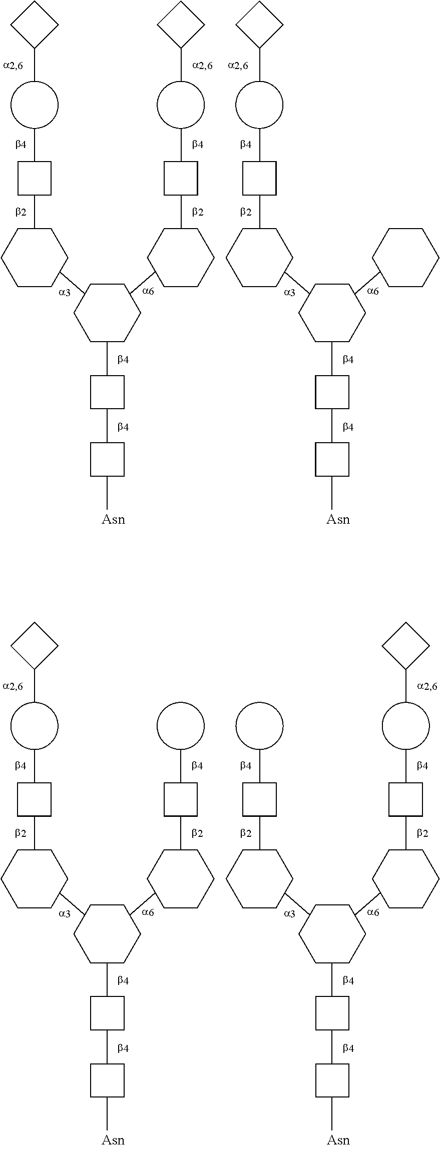
wherein the diamond represents a sialic acid residue, the empty circle represents a galactose residue, the square represents an N-acetylglucosamine residue and the hexagon represents a mannose residue, and wherein the Asn is an Asn of an N-linked glycosylation consensus sequence in a variable domain of the monoclonal antibody.

17. The monoclonal antibody of claim 1, comprising one or more of the following structures: 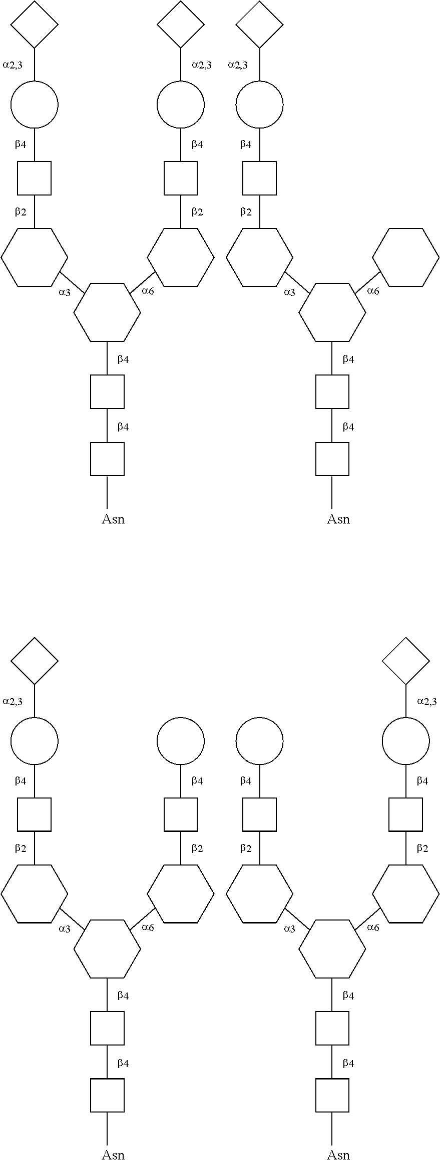
wherein the diamond represents a sialic acid residue, the empty circle represents a galactose residue, the square represents an N-acetylglucosamine residue and the hexagon represents a mannose residue, and wherein the Asn is an Asn of an N-linked glycosylation consensus sequence in a variable domain of the monoclonal antibody.

18. The monoclonal antibody of claim 1, comprising one or more of the following structures:

| | Oxford/Unifi | Short IgG | Symbol Nomenclature for Glycan |
|---|---|---|---|
| | nomenclature¹ | nomenclature² | structure³ |
| 1 | M3 | Man3 | 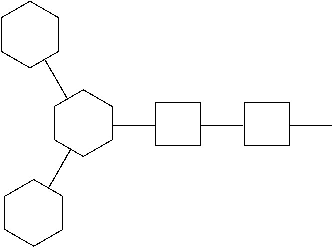 |
| 2 | A1 | G0-N | 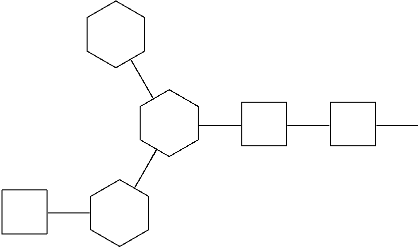 |
| 3 | A1G1 | G1-N | 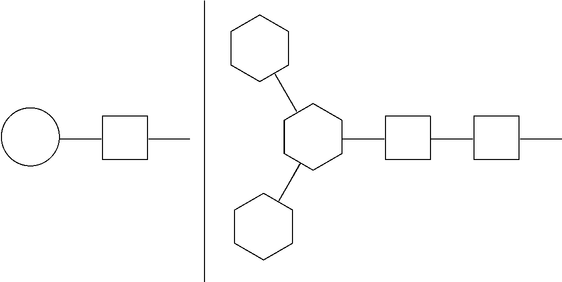 |
| 4 | A1[3]G(4)1 | G1-N | 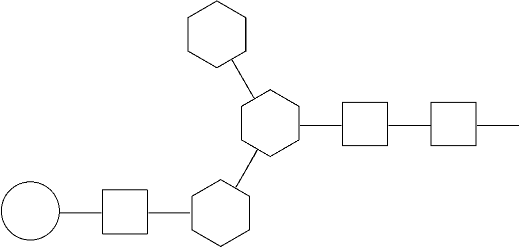 |
| 5 | A2 | G0 | 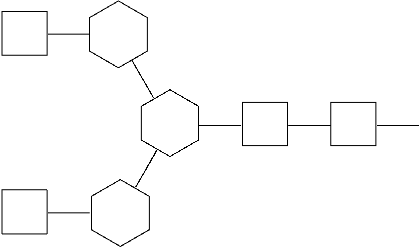 |
| 6 | A2[6]G(4)1 | G1(6′) | 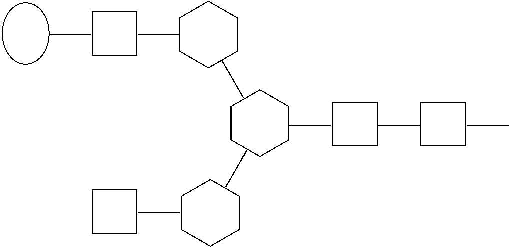 |
| 7 | A2[3]G(4)1 | G1(3′) | 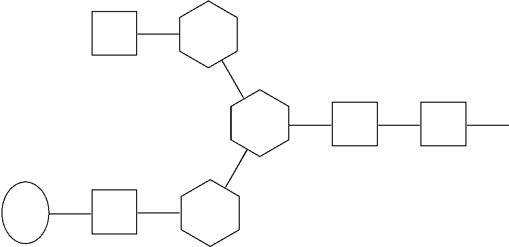 |
| 8 | A2G1 | G1 | 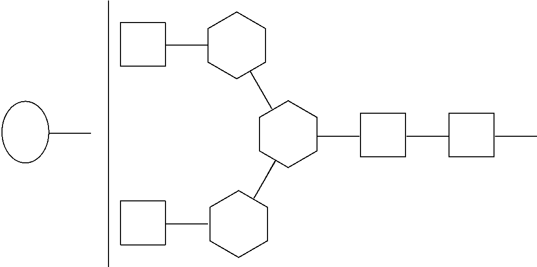 |
| 9 | A1G1S1 | G1S1-N | 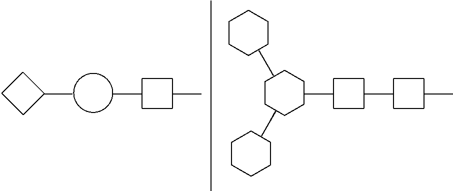 |
| 10 | A1[3]G(4)1S1 | G1S1-N | 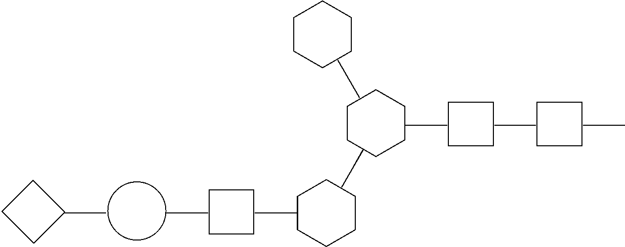 |
| 11 | A2G(4)2 | G2 | 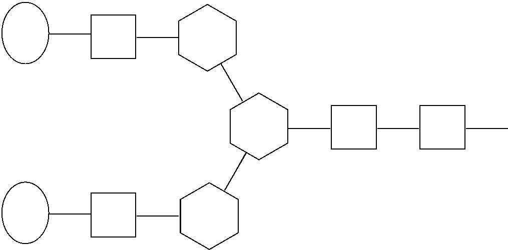 |
| 12 | A2G1S1 | G1S1 | 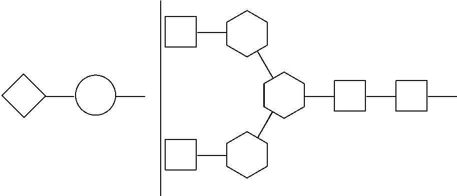 |
| 13 | A2[6]G(4)1S(6)1 | G1S1 | 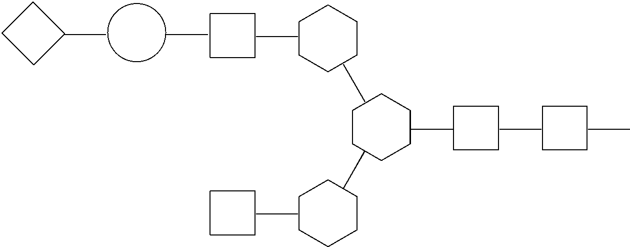 |
| 14 | A2[3]G(4)1S(6)1 | G1S1 | 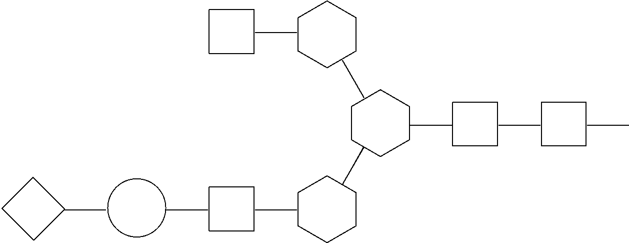 |
| 15 | A2G2S1 | G2S1 | 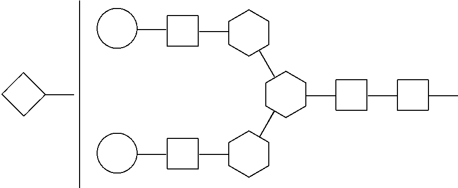 |
| 16 | A2G2S2 | G2S2 | 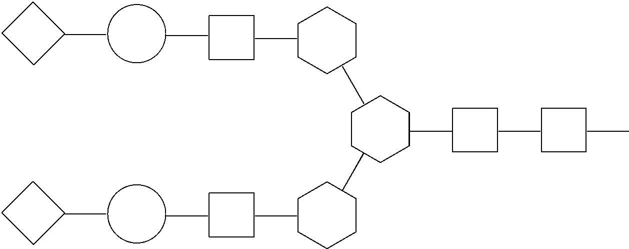 |
| 17 | A2G(4)2S(6,6)2 | G2S2 | 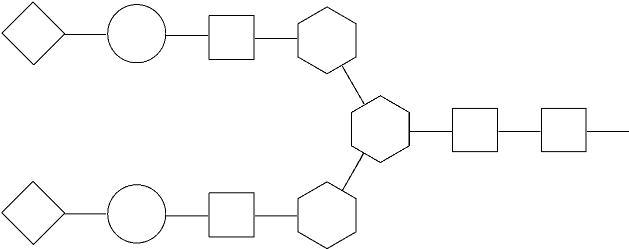 |

¹Mx: number (x) of residues within the oligomannose series;
Ax: number (x) of antennae;
F: core fucose;
Gx: number (x) of galactoses;
B: bisecting GlcNAc;
S: number (x) of sialic acids.
Note:
Linkage information is given in ( ) parentheses if applicable, e.g. F(6)A2 − α1-6 linked fucose. A2G1S1(6) − α2-6 linked sialic acid. Brackets [x] before G indicate which arm of the mannosyl core is galactosylated e.g. [3]G1 indicates that the galactose is on the antenna of the α1-3 mannose.
²This typically with IgG associated naming system indicates the presence of core fucose, the number of galactoses and the presence of biantennary glycans. It is limited in the number of structures and linkages it can describe but is often used for simplicity.
³Hexagon represents mannose (Man), white square is N-acetyl glucosamine (GlcNAc), white circle is galactose (Gal), white diamond is sialic acid, N-acetyl neuraminic acid (Neu5Ac).
wherein the diamond represents a sialic acid residue, the empty circle represents a galactose residue, the square represents an N-acetylglucosamine residue and the hexagon represents a mannose residue; and wherein the reducing end is on Asn of an N-linked glycosylation consensus sequence in the monoclonal antibody.

19. The monoclonal antibody of claim 11, wherein:
a. the anti-TNFα antibody has an antibody-dependent cell mediated cytotoxicity (ADCC) activity that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than that of the same anti-TNFα antibody having a different glycosylation profile; or
b. the anti-TNFα antibody has an antibody-dependent cell mediated cytotoxicity (ADCC) activity against primary inflammatory target cells that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than that of the same anti-TNFα antibody having a different glycosylation profile; or
c. the anti-TNFα antibody has a reduced (lower) immunogenicity that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower than that of the same anti-TNFα antibody having a different glycosylation profile; or
d. the anti-TNF antibody has an increase wound healing M2 macrophages induction activity that is at least 10%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than that of the same anti-TNFα antibody having a different glycosylation profile.

20. (canceled)

21. (canceled)

22. (canceled)

23. A Leishmania host cell genetically engineered as described in Example X comprising the monoclonal antibody of claim 1.

24. (canceled)

25. A method for making a monoclonal antibody comprising culturing the Leishmania host cell of claim 23 and isolating the monoclonal antibody.

26. A monoclonal antibody produced by the method of claim 25.

27. A pharmaceutical composition comprising the monoclonal antibody of claim 1 and a pharmaceutically acceptable carrier.

28. A method of treating or preventing a disease in a patient comprising administering to the patient the monoclonal antibody of claim 1.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. A single dosage form of a monoclonal antibody of claim 1, wherein the single dosage form consists of about 2 mg, about 5 mg, about 7 mg, about 10 mg, about 12 mg, about 15 mg, about 18 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg of the anti-TNFα antibody.

43. (canceled)

44. (canceled)


SEQUENCE LISTING (3 sequences)

SEQ ID NO: 1 - protein, 357 aa, Homo sapiens
  Human UDP-GlcNAc Transporter (Uniprot Q9Y2D2), N-term 3xHA tag
MYPYDVPDYAYPYDVPDYAYPYDVPDYAGGSGGFANLKYVSLGILVFQTTSLVLTMRYSRTLKEEGPRYLSSTAVVVAELLKIMACILLVYKDSKCSLRALNRVLHDEILNKPMETLKLAIPSGIYTLQNNLLYVALSNLDAATYQVTYQLKILTTALFSVSMLSKKLGVYQWLSLVILMTGVAFVQWPSDSQLDSKELSAGSQFVGLMAVLTACFSSGFAGVYFEKILKETKQSVWIRNIQLGFFGSIFGLMGVYIYDGELVSKNGFFQGYNRLTWIVVVLQALGGLVIAAVIKYADNILKGFATSLSIILSTLISYFWLQDFVPTSVFFLGAILVITATFLYGYDPKPAGNPTKA

SEQ ID NO: 2 - protein, 344 aa, Homo sapiens
  Beta-galactoside alpha-2,6-sialyltransferase 1 (Uniprot P15907), aa89-406, N-terminal synthetic signal peptide
MIASSVRHAVILLLVAVAMMVNNVIAPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 3 - protein, 579 aa, Mouse
  CMP-Neu5Ac transporter (Q61420.2), GFP fusion
MAPARENVSLFFKLYCLTVMTLVAAAYTVALRYTRTTAEELYFSTTAVCITEVIKLLISVGLLAKETGSLGRFKASLSENVLGSPKELAKLSVPSLVYAVQNNMAFLALSNLDAAVYQVTYQLKIPCTALCTVLMLNRTLSKLQWISVFMLCGGVTLVQWKPAQATKVVVAQNPLLGFGAIAIAVLCSGFAGVYFEKVLKSSDTSLWVRNIQMYLSGIVVTLAGTYLSDGAEIQEKGFFYGYTYYVWFVIFLASVGGLYTSVVVKYTDNIMKGFSAAAAIVLSTIASVLLFGLQITLSFALGALLVCVSIYLYGLPRQDTTSIQQEATSKERIIGVGGSGGMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK